(19) United States
(12) Patent Application Publication
(10) Pub. No.: US 2025/0100984 A1
(43) Pub. Date: Mar. 27, 2025

(54) SELECTIVE PARTIAL REDUCTION OF ESTERS USING GROUP IV TRANSITION METAL CATALYSTS AND USES THEREOF

(71) Applicant: BAYLOR UNIVERSITY, Waco, TX (US)

(72) Inventors: Liela A. Romero, Waco, TX (US); Rebecca A. Kehner, Waco, TX (US); Weiheng Huang, Waco, TX (US); Jack Russo, Waco, TX (US)

(73) Assignee: BAYLOR UNIVERSITY, Waco, TX (US)

(21) Appl. No.: 18/894,623

(22) Filed: Sep. 24, 2024

Related U.S. Application Data
(60) Provisional application No. 63/540,205, filed on Sep. 25, 2023.

(51) Int. Cl. C07D 295/027; C07C 45/41; C07C 209/16

(52) U.S. Cl. CPC C07D 295/027; C07C 45/41; C07C 209/16

(57) ABSTRACT

Methods described herein relate to a partial reduction of esters to aldehydes or nitrogen-containing products facilitated by Group IV transition metal catalysts, using hydrosilanes as the reductant. The method allows the product to be preserved at the aldehyde oxidation level rather than over-reduction to the corresponding alcohol and can result in the formation of value-added recycled monomers. Methods described herein can be used for the direct catalytic chemical upcycling of polyester plastic waste through depolymerization transformations.

This application claims priority to U.S. Provisional Patent Application No. 63/540,205, entitled “Selective Partial Reduction of Esters Using Group IV Transition Metal Catalysts and Uses Thereof,” filed Sep. 25, 2023, the entire contents of which are hereby incorporated by reference.

### BACKGROUND

The present disclosure relates to methods for direct conversion of esters to imines, enamines, or other nitrogen-containing products, or, if hydrolysis is employed, to aldehydes facilitated by Group IV transition metal catalysts.

The ester functional group is ubiquitous among organic compounds, including synthetic materials (such as plastics, fabrics, etc.), biomolecules, and natural products. Semi-reductive transformations of esters remain underdeveloped. A direct catalytic semi-reductive imination or enamination of esters would offer an alternative approach to synthetic strategies that require sequential reduction and condensation steps.

Analogously, the conversion of esters to aldehydes through partial reduction is a widely desired transformation in organic synthesis. The conversion of esters to aldehydes is typically carried out by either full reduction to an alcohol followed by reoxidation to an aldehyde, or by pre-activation followed by reduction. These methods are undesirable due to the use of pyrophoric reagents, the formation of difficult emulsions, unreliability, and/or the use of multiple steps which makes them expensive to carry out. Thus, there is ongoing interest in developing safe and reliable procedures for the partial reduction of esters to aldehydes, especially those that largely avoid the production of alcohol contaminants resulting from over-reduction.

The catalytic partial reduction of esters has direct applications for the chemical recycling of accumulated plastic waste as well as other areas. For example, polyethylene terephthalate (PET) is the most common commercial polyester recyclable, often found in beverage bottles. This material is typically depolymerized to carboxylic acid derivatives (via glycolysis, methanolysis, aminolysis) or to regenerate terephthalic acid via hydrolysis. The selective reduction of esters to aldehydes is difficult due to the greater propensity of aldehydes to undergo further reduction to alcohols. Halting ester reduction at the aldehyde oxidation state is consequently challenging.

### SUMMARY

The present disclosure pertains to a method for selective partial reduction of esters to aldehydes or nitrogen-containing products facilitated by Group IV transition metal catalysts using hydrosilanes as the stoichiometric reductant. In particular, the present disclosure relates to highly selective methods for the interconversion of esters to aldehydes or nitrogen-containing compounds, such as imines, enamines, hydrazones, N-heterocycles, or amines, through an amine-intercepted zirconocene hydride (ZrH)-catalyzed reduction.

Preferred embodiments of methods described herein employ an inexpensive zirconium catalyst in combination with hydrosilanes and simple unprotected amines. A variety of aryl, benzylic, and aliphatic esters are directly transformed to imines or enamines in up to 99% yield or aldehydes in up to 84% yield, with little-to-no over-reduction to the corresponding alcohols. The preparation of other nitrogen-containing products via single-flask multi-stage reactions is also disclosed. The applicability of this catalytic protocol toward the chemical upcycling of polyester plastic waste is demonstrated through a series of novel depolymerization reactions employing polyester-based plastics and fibers.

FIG. 1 shows different strategies for the conversion of esters to aldehydes, imines and enamines, including (a) traditional strategies with poor step and redox economy, (b) a disclosed strategy for amine-mediated interception of zirconocene hemiaminals. FIG. 2 shows a preferred strategy, as described herein, for the partial reduction of esters using a ZrH-catalyzed direct strategy.

Although prior protocols using group IV transition metals result in some degree of over-reduction to the corresponding alcohol, the protocol described herein utilizes a trapping mechanism to protect the product at the aldehyde oxidation state.

Interest in zirconocene hydride (ZrH) catalysis has prompted the exploration of new methodologies concerning the functional group interconversion of carbonyl-containing molecules. Until now, manifolds employing ZrH reagents in either stoichiometric or catalytic quantities have exclusively resulted in the full reduction of esters to alcohols. Earlier work regarding the ZrH-catalyzed reduction of carbonyls using hydrosilanes showcased this selectivity, as did a later report by others using an analogous strategy but employing Schwartz's reagent (Cp₂ZrHCl) directly. In both studies aldehyde intermediates were not observed, even in trace quantities, as these intermediates are more susceptible to reduction than the starting materials themselves.

The transaminative semi-reduction of tertiary amides to imines and enamines has been disclosed, as shown in FIG. 1(b). Key insights from accompanying mechanistic studies suggest the nucleophilic interception of zirconocene hemiaminal intermediates by an exogenous unprotected amine. This platform can be extended by analogy to the partial reduction of esters. This would instead promote formation of imines or enamines as a protective trap for the aldehyde oxidation level. This mechanistic distinction likewise delivers a synthetic handle for the direct conversion of esters to nitrogen-containing products.

The application of this protocol to the depolymerization of commercial polyester materials results in the formation of value-added recycled imine or aldehyde monomers. This approach is distinct from traditional polyester depolymerization approaches, which instead yield alcohol, ester, amide or carboxylic acid monomers. The methods described herein may be useful in other areas as well, such as late stage drug modification protocols.

### BRIEF DESCRIPTION OF THE DRAWINGS

FIG. 1 shows (a) schematics of traditional pathways used for conversion of esters to aldehydes, imines and enamines in comparison to the present strategy, and (b) a scheme for amine-mediated interception of zirconocene hemiaminals.

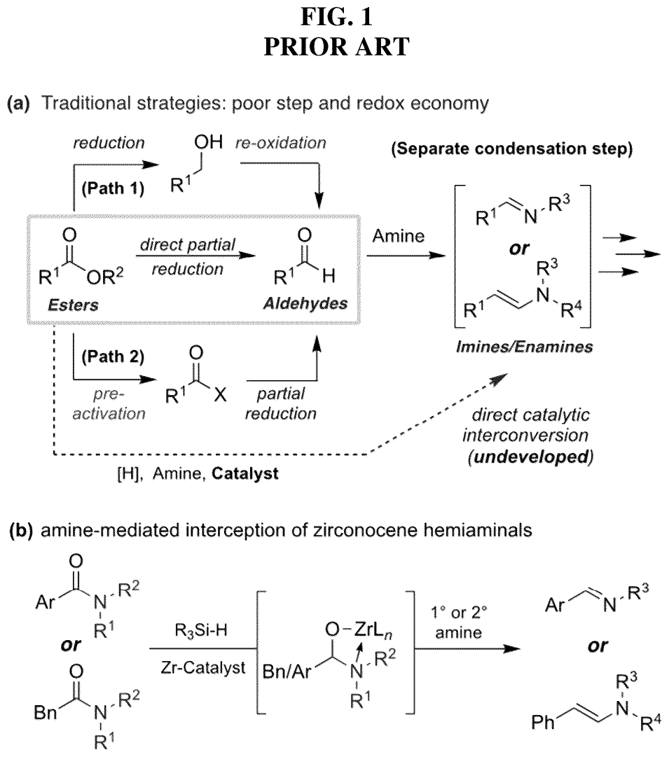

FIG. 2 shows a scheme for the partial reduction of esters via zirconocene hydride catalysis according to preferred embodiments described herein.

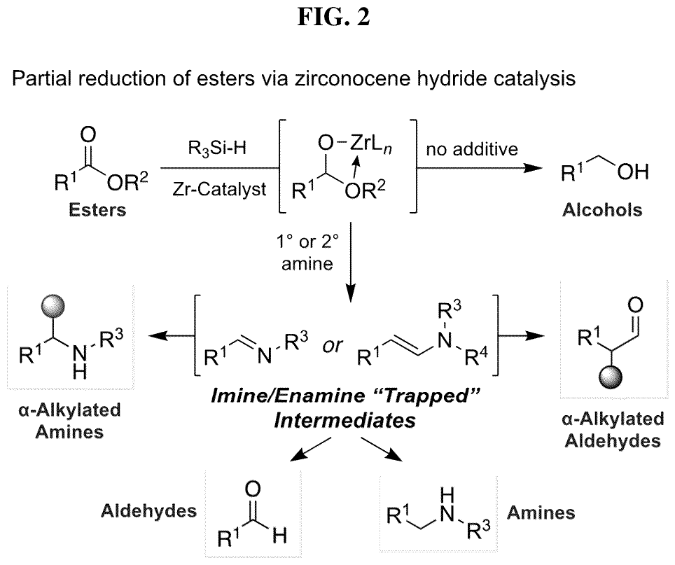

FIG. 3 shows exemplary schemes and results for optimizing of ZrH-catalyzed reduction of esters according to preferred embodiments described herein.

FIG. 4A shows an exemplary scheme for catalytic partial reduction of esters to aldehydes, imines, enamines and hydrazones under different sets of conditions, with representative substrates and yields of the products for each substrate.

FIG. 4B shows a scheme and results for alkylative amination via nucleophilic trapping, a scheme and result for semi-reductive aklylation via electrophilic trapping, and results for single-flask reductive amination of esters.

FIG. 5 shows a scheme and results for screening of amines for the semi-reduction of methyl 4-chlorobenzoate.

FIG. 6 shows a scheme and results of control experiments for the semi-reduction of methyl 4-chlorobenzonate.

FIG. 7 shows a scheme and results for testing amidation in the presence of 20 mol % DEMS.

FIG. 8 shows a scheme and results for screening of metallocenes for the semi-reduction of methyl 4-chlorobenzoate.

FIG. 9 shows a scheme and results for extended optimization of ZrH-catalyzed reduction of esters.

FIG. 10 shows a scheme and results of extended optimization for the semi-reduction of methyl 2-(4-bromophenyl)acetate.

FIG. 11A shows a scheme for depolymerization of polyethylene terephthalate from various sources to produce an aldehyde.

FIG. 11B shows a scheme for depolymerization of polyethylene terephthalate without hydrolysis.

FIG. 11C shows a scheme for depolymerization of poly(ethylene succinate).

FIG. 12A shows a plausible mechanistic pathway involving nucleophilic interception of Zr-acetal.

FIG. 12B shows a plausible mechanistic pathway involving β-alkoxide elimination.

FIG. 12C shows chemoselectivity comparison for various Zr catalysts.

FIG. 13 shows schemes for general procedures A, B, and C for preparation of starting materials.

### DETAILED DESCRIPTION OF PREFERRED EMBODIMENTS

The present disclosure relates to methods for partial reduction of esters that use Group IV metal catalysts and access imine and enamine “trapped” intermediates to produce aldehydes and nitrogen-containing products.

Preferred embodiments described herein relate to a method for partial reduction of esters comprising reacting an ester with a Group IV metal catalyst and a silane in the presence of an amine to produce an aldehyde or a nitrogen-containing compound such as an imine, enamine, hydrazone or amine. In further preferred embodiments, the ester is an aryl ester or an aliphatic ester and the Group IV metal is zirconium, titanium or hafnium. Additional preferred Group IV metal catalysts include zirconocene dichloride (C₁₀H₁₀Cl₂Zr or Cp₂ZrCl₂) and zirconocene hydrochloride (C₁₀H₁₁ClZr or Cp₂ZrHCl). Preferred silanes include dimethoxy(methyl)silane (DMMS), diethoxy(methyl)silane (DEMS), and polymethylhydrosiloxane (PMHS), as well as other hydrosilanes, including di-, oligo- and polysiloxanes, as well as arylsilanes and trialkoxysilanes. Preferred amines include primary or secondary amines, including but not limited to n-butylamine and piperidine.

Preferred embodiments of methods enabled by ZrH catalysis described herein lead to a series of highly selective and novel reductive transformations of esters. The interrupted catalytic reduction of esters via addition of simple unprotected amines results in the formation of imine and enamine “trapped” intermediates thereby preserving the intermediate oxidation level. The exceptional selectivity of this catalytic strategy was demonstrated through seminal semi-reductive iminations and enaminations of esters in up to 99% yield. Analogously, either monomeric esters or polyester materials afforded aldehydes in high yields and with excellent chemoselectivity. α-Alkylated aldehydes and amines are also accessible through single-flask operations via electrophilic or nucleophilic interception of intermediates. Further, the reductive amination of esters using primary and secondary amines has been established.

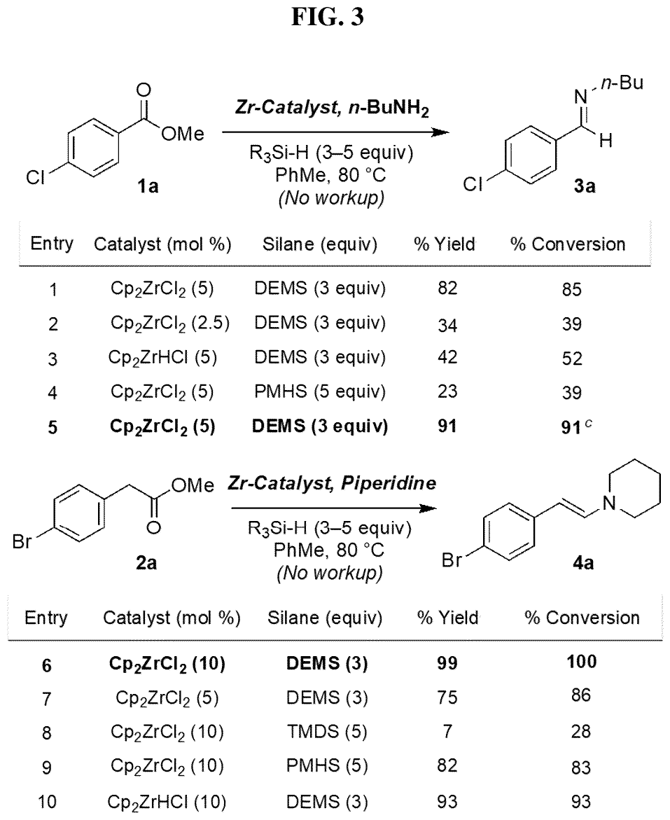

Examples of preferred embodiments of the highly selective interconversion of esters to imines, enamines, aldehydes, hydrazones or amines through an amine-intercepted zirconocene hydride (ZrH)-catalyzed reduction are discussed in the examples below and illustrated in FIGS. 3-10.

As a direct application toward the chemical recycling of accumulated plastic waste, this catalytic protocol was leveraged for new modes of polyester depolymerizations. The catalytic depolymerization of polyester plastics to access versatile chemical feedstocks at an oxidation state in-between that of their original carboxylic acid starting monomers and the fully reduced alcohol monomers would be an unconventional form of plastic upcycling. Polyethylene terephthalate (PET), often found in plastic beverage bottles, containers, and fabrics, represents the most common polyester recyclable.

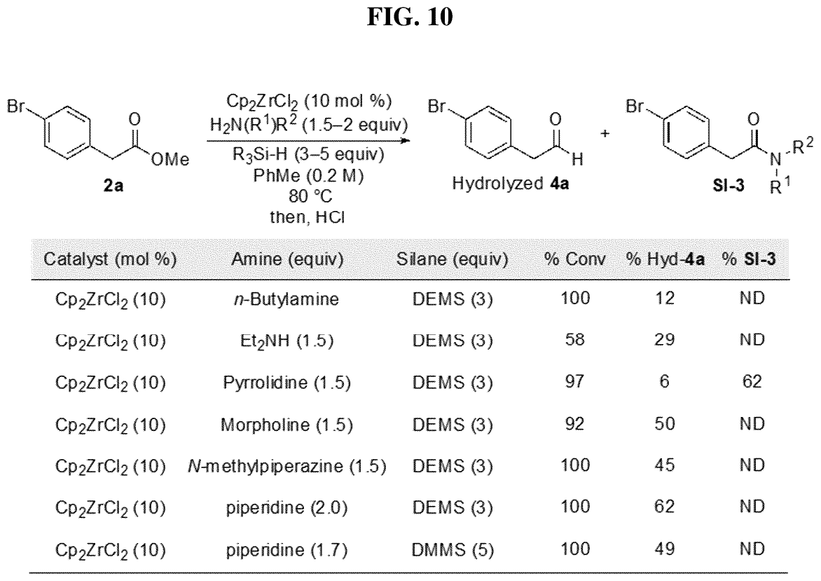

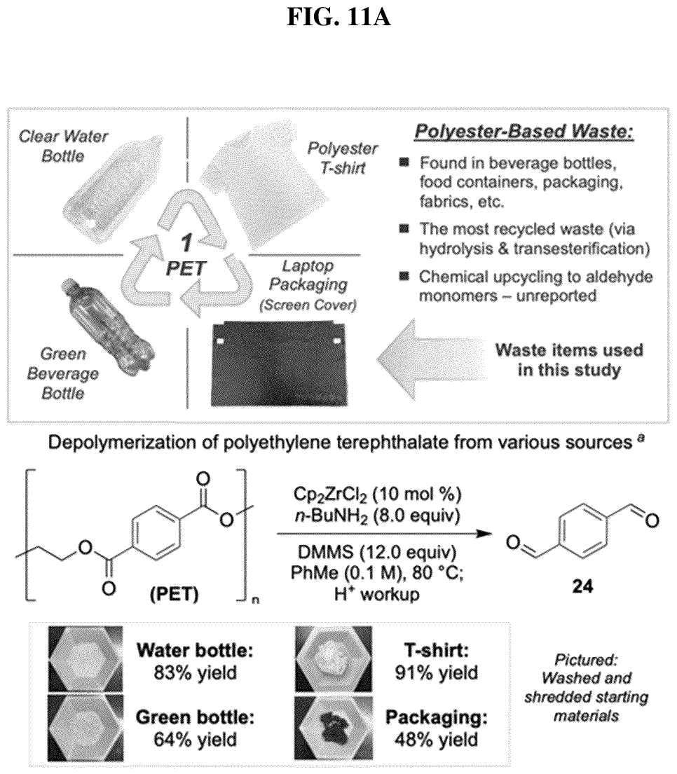

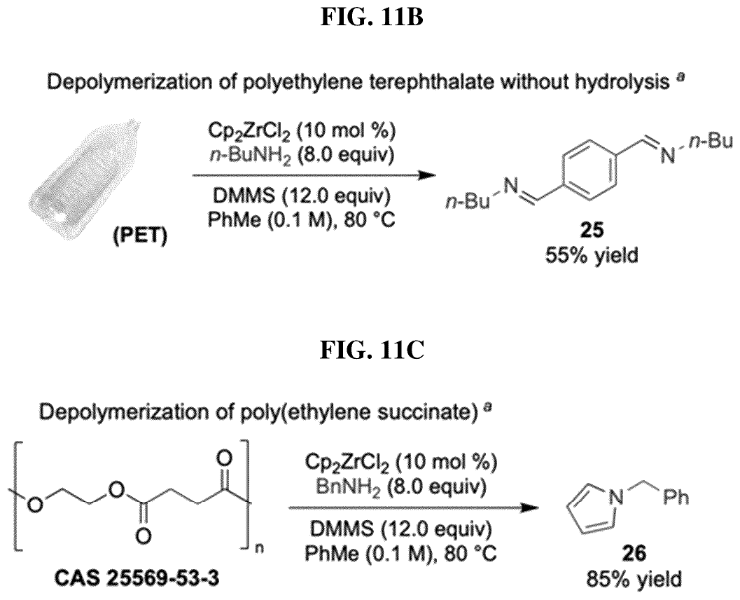

FIG. 11A demonstrates the conversion of an assortment of post-consumer PET wastes to terephthalaldehyde, a versatile building block used for the preparation of small molecules, polymers, and other porous and nonporous materials with wide-ranging uses (ligands, dyes, sensors, liquid crystals, thin films, re-healable thermosets, etc.). Utilizing modified semi-reduction conditions, as discussed more fully in the examples below, PET plastic pieces obtained directly from a single use water bottle or a green beverage bottle afforded terephthalaldehyde (24) in 83% and 64% yields, respectively. The depolymerization of polyester fabric was also demonstrated and discussed in the examples below. Whether using PET sourced from a 100% polyester t-shirt or dyed fibers belonging to packaging waste from a recent laptop purchase (fabric screen protector insert), the catalytic reduction produced dialdehyde 24 in reasonable quantities (91% and 48% yield respectively).

The strength of this strategy, however, lies in the ability to directly convert polyester waste to nitrogen-containing building blocks, offering a unique strategy for the repurposing of this plastic. For example, when PET was subjected to the standard depolymerization conditions without hydrolytic workup, diimine 25 was isolated in 55% yield (FIG. 11B). Further, poly(ethylene) succinate, an aliphatic polyester, underwent a novel depolymerization-cyclization sequence under similar reaction conditions (FIG. 11C). When the catalytic protocol was carried out using benzylamine, depolymerization occurred with concomitant Paal-Knorr-type cyclization to furnish pyrrole 26 in 85% isolated yield.

Accordingly, preferred embodiments disclosed herein include methods for partial reduction of an ester to an aldehyde or nitrogen-containing compound, including steps of preparing a reaction mixture comprising the ester, a Group IV metal catalyst, a silane, and an amine, allowing the reaction mixture to undergo a partial reduction reaction, and isolating an aldehyde or nitrogen-containing compound from the reaction mixture. The Group IV metal catalyst may comprise zirconium, titanium, or hafnium and, in additional preferred embodiments, the Group IV metal catalyst can be zirconocene dichloride or zirconocene hydrochloride. The silane can be dimethoxy(methyl)silane (DMMS), diethoxy(methyl)silane (DEMS), or polymethylhydrosiloxane (PMHS) or other hydrosilanes, including di-, oligo- and polysiloxanes, as well as arylsilanes and trialkoxysilanes. The amine added to the reaction mixture can be a primary or secondary amine such as n-butylamine or piperidine. In some embodiments, the ester subjected to partial reduction is a polyester. In additional preferred embodiments, the ester is an aryl ester, the Group IV metal catalyst is zirconocene dichloride or zirconocene hydrochloride, the silane is dimethoxy(methyl)silane (DMMS) or diethoxy(methyl)silane (DEMS), and the amine is n-butylamine. In further preferred embodiments, the ester is an aliphatic ester, the Group IV metal catalyst is zirconocene dichloride or zirconocene hydrochloride, the silane is diethoxy(methyl)silane (DEMS) or polymethylhydrosiloxane (PMHS), and the amine is piperidine. The reaction mixture may further comprise anhydrous PhMe.

In additional preferred embodiments, the methods for partial reduction of an ester to an aldehyde or nitrogen-containing compound also include a step of placing the reaction mixture under a nitrogen environment prior to allowing the reaction mixture to undergo a partial reduction reaction. In further preferred embodiments, the methods may also include a step of maintaining the reaction mixture at a temperature of 80° C. while the partial reduction reaction occurs.

Additional preferred embodiments described herein include a method for depolymerization of a polyester to an aldehyde or nitrogen-containing compound, where the method includes the steps of preparing a reaction mixture comprising the polyester, a Group IV metal catalyst, a silane, and an amine, allowing the reaction mixture to undergo a partial reduction reaction, and isolating an aldehyde or nitrogen-containing compound from the reaction mixture. In preferred embodiments, the Group IV metal catalyst is zirconocene dichloride or zirconocene hydrochloride, the silane is dimethoxy(methyl)silane (DMMS), diethoxy(methyl)silane (DEMS), or polymethylhydrosiloxane (PMHS), and the amine is n-butylamine or piperidine.

In all preferred embodiments, the methods for partial reduction or depolymerization produce aldehydes or nitrogen-containing compounds that include imines, enamines, hydrazones, N-heterocycles, or amines, depending on the starting material and the conditions selected.

### EXAMPLES

### Materials and Methods

General procedural information. Reactions were carried out under an atmosphere of nitrogen in flame-dried glassware with magnetic stirring unless otherwise noted. Anhydrous toluene (PhMe) and anhydrous tetrahydrofuran (THF) were purchased from Fisher Chemical, stored in Apache Stainless solvent kegs, and dried by passage under argon pressure through columns packed with alumina and R3-15 (PhMe) or alumina (THF). Liquids and solutions were transferred via syringe. Bis(cyclopentadienyl) zirconium dichloride (Cp₂ZrCl₂) was purchased from Sigma Aldrich and stored in a desiccator. Schwartz's Reagent (Cp₂ZrHCl) was purchased from Strem Chemicals and stored in a nitrogen-atmosphere glovebox. Diethoxy(methyl)silane (DEMS) was purchased from TCI Chemicals and stored at 0° C. Polymethylhydrosiloxane (PMHS) was purchased from Sigma Aldrich and stored at 0° C. n-Butylamine was purchased from Oakwood Chemicals and distilled prior to use. Piperidine was purchased from Sigma Aldrich. All other commercially available materials were used as received. Medium pressure chromatography purifications were conducted with the assistance of a Teledyne NextGen 300 Chromatography System unless otherwise noted. Reusable cartridges (4 g-24 g) were purchased from Biotage and disposable GOLD cartridges (4 g-24 g) were purchased from Teledyne Isco. Manual packing of reusable cartridges was performed using P60 SiliaFlash silica gel (40-63 μm, 230-400 mesh). Activated neutral aluminum oxide (Brockmann Grade II, 58 Å) was purchased from Thermo Fisher Scientific. Organic solutions were concentrated with the aid of a Buchi rotary evaporator equipped with a Buchi vacuum regulator. Isolated yields are reported for products of ≥96% purity, unless otherwise noted.

General analytical information. All reactions were monitored by thin-layer chromatography using Silicycle SiliaPlate pre-coated plates (0.25 mm) and visualized with UV light and/or KMnO₄ stain. All nuclear magnetic resonance (NMR) spectra were recorded on a Bruker Avance III HD 600 MHz, Bruker Avance III HD 400 MHz, or Agilent VNMRS 500 MHz instrument. ¹H and ¹³C spectra were recorded in CDCl₃ or toluene-d8 and chemical shifts are reported in δ units, parts per million (ppm), relative to residual chloroform or toluene in the deuterated solvent (for CDCl₃: 7.26 ppm for ¹H NMR and 77.16 ppm for ¹³C NMR; for toluene-d8: 2.09 ppm for ¹H NMR). Coupling constants (J) are reported in Hertz (Hz). Infrared (IR) spectra were recorded on a Bruker Platinum-ATR IR spectrometer using a diamond window and reported in terms of frequency of absorption (cm⁻¹). High resolution mass spectrometry (HRMS) data was obtained using electro-spray ionization (ESI) using a Thermo LTQ Orbitrap Q-Exactive instrument. Low-resolution mass spectrometry (LRMS) data was collected using an Agilent 8890 gas chromatography (GC) system equipped with a 5977B series inert mass selective detector. Analytical high performance liquid chromatography (HPLC) analysis was performed using an Agilent 1260 Infinity II instrument equipped with commercial columns obtained from Chiralcel (Daicel Corporation) with the following specifications: OD-H (4.6 mm I.D.×250 mm L, particle size 5 μm, and part no. 14325), and OJ-H (4.6 mm I.D.×250 mm L, particle size 5 μm, and part no. 17325).

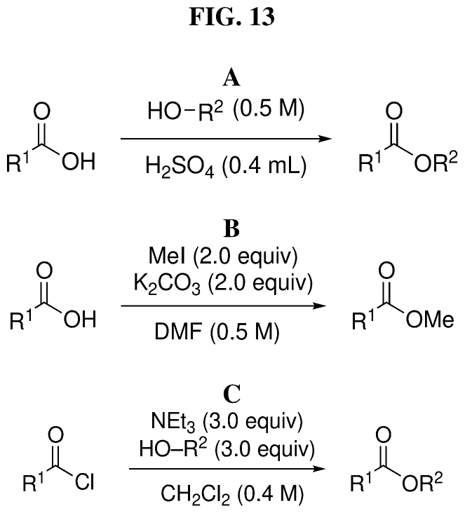

Preparation of starting materials. Example 6 and FIG. 13 include additional details regarding preparation of starting materials.

### Example 1

### General Procedure A: Reduction of Aryl and Alkenyl Esters

Cp₂ZrCl₂ (14.6 mg, 5.0 mol %) and solid starting materials (1.0 mmol) were weighed into a flame-dried 20×125 mm reaction tube equipped with a magnetic stir bar. The reaction tube was capped with an open top screw cap equipped with a Teflon-lined silicon septum and sealed with electrical tape. The reaction tube was then evacuated and backfilled with nitrogen (this process was repeated to a total of three times). The solids were dissolved in 2.5 mL of anhydrous PhMe (0.4 M). Liquid substrates were injected at this point. DEMS (480.6 μL, 3.0 equiv) was injected into the reaction mixture, followed by the injection of distilled n-butylamine (168.0 μL, 1.7 equiv) using a Hamilton gastight glass microsyringe. n-Butylamine was stored at 0° C. and under an atmosphere of N₂ after distillation. The amine was distilled approx. every two weeks, or sooner if the solution was no longer colorless in appearance The septum of the reaction tube was sealed with wax and the reaction solution was stirred at 400 rpm at 80° C. for 19-48 h. To obtain aldehyde products, the reaction mixture was then quenched with 5 mL of 1 M HCl and stirred at 80° C. for approx. 1 hour. The solution was diluted with ca. 5 mL of Et₂O and ca. 5 mL H₂O. The aqueous layer was washed three times with ca. 5 mL Et₂O. The combined organic washes were dried over MgSO₄, vacuum filtered, and concentrated in vacuo with the aid of a rotary evaporator.

### General Procedure B: Reduction of Aliphatic (Enamine-Forming) Esters

Cp₂ZrCl₂ (29.2 mg, 10.0 mol %) and solid starting materials (1.0 mmol) were weighed into a flame dried 20×125 mm reaction tube equipped with a magnetic stir bar. The reaction tube was capped with an open top screw cap equipped with a Teflon-lined silicon septum and sealed with electrical tape. The reaction tube was then evacuated and backfilled with nitrogen (this process was repeated to a total of three times). The solids were dissolved in 2.5 mL of anhydrous PhMe (0.4 M). Piperidine (148.1 μL, 1.5 equiv) was injected into the reaction mixture. Liquid starting materials were injected at this point, followed by the injection of DEMS (480.6 μL, 3.0 equiv) using a Hamilton gastight glass microsyringe. The septum of the reaction tube was sealed with wax and the reaction solution was stirred at 700 rpm at 80° C. for 17-24 h. To obtain aldehyde products, the reaction mixture was then quenched with 5 mL of 1 M HCl and stirred at 80° C. for approx. 1 hour. The solution was diluted with ca. 5 mL of Et₂O and ca. 5 mL H₂O. The aqueous layer was washed three times with ca. 5 mL Et₂O. The combined organic washes were dried over Na₂SO₄, vacuum filtered, and concentrated in vacuo with the aid of a rotary evaporator.

Initial studies employing zirconocene dichloride (Cp₂ZrCl₂) as a pre-catalyst in combination with hydrosilanes as the reductant. Results of reaction optimization are shown in FIG. 3. In FIG. 3, Entries 1-4: Reactions were carried out under a N₂ atmosphere using 0.2 mmol of 1a and 1.7 equiv n-butylamine in anhydrous PhMe (0.4 M) for a duration of 18 h; Entries 6-10: Reactions were carried out under a N₂ atmosphere using 0.2 mmol of 2a and 1.5 equiv piperidine in anhydrous PhMe (0.4 M) for a duration of 18-23 h. Yields were determined by ¹H NMR spectroscopy of the crude reaction mixture, using mesitylene as an internal standard. Entry 5 was carried out using 1 mmol 1a for 21 h instead.

Initial attempts to reduce aryl ester 1a with diethoxy(methyl)silane (DEMS) in the absence of an amine either resulted in over-reduction to the alcohol or unreacted starting material. We then carried out the reduction in the presence of n-butylamine (entry 1). Product selectivity diverged, favoring the formation of imine 3a in 82% yield. Conversion of 1a was diminished when catalyst loading was decreased or when Cp₂ZrHCl was employed as the catalyst (entries 2 and 3). Replacing DEMS with polymethylhydrosiloxane (PMHS) likewise resulted in lower conversion and yield (entry 4). Finally, the yield of 3a was improved upon simply increasing reaction time from 18 to 21 hours (entry 5). Notably, <5% of alcohol was observed throughout the course of these optimization studies.

After identifying the optimal reaction conditions for the semi-reductive imination of aryl esters, aliphatic ester 2a was considered. A secondary amine was considered for use to promote the formation of an enamine. Upon exploration of various amine additives, piperidine proved to be the optimal amine, quantitatively furnishing enamine 4a (entry 6). Decreasing the catalyst loading to 5 mol % provided the desired product in a synthetically useful, but lower yield (entry 7). Replacing DEMS with tetramethyldisiloxane (TMDS) proved ineffective; however, PMHS promoted the transformation of 2a, albeit with a slightly lower yield of 82% (entries 8 and 9). This hydrosilane, a byproduct of the silicon industry, is an especially appealing reductant due to its low cost and safety profile. Lastly, Cp₂ZrHCl facilitated the semi-reduction with similar efficiency as Cp₂ZrCl₂ (entry 10).

### Example 2

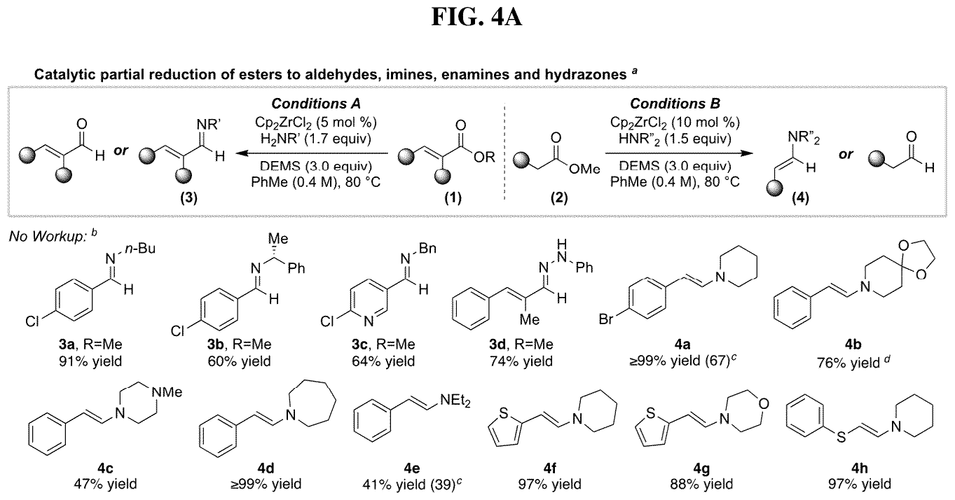

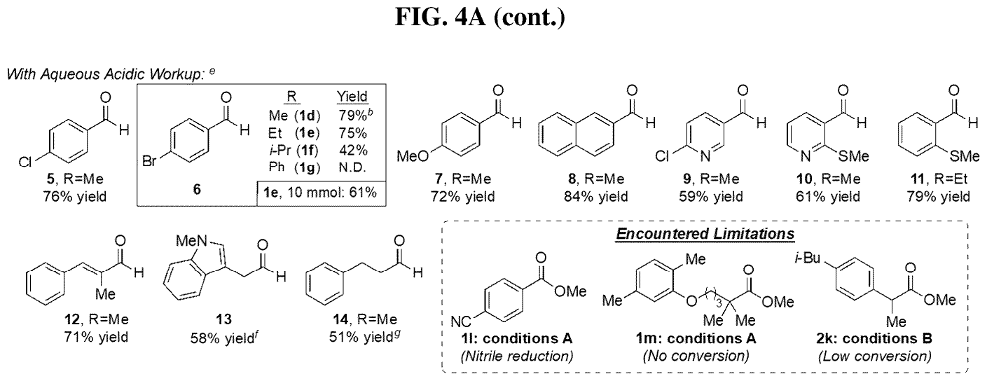

FIG. 4A shows an exemplary scheme for catalytic partial reduction of esters to imines, enamines, or aldehydes, along with the structures of imine and enamine products obtained. In. FIGS. 4A and 4B, [a] Reactions were carried out under an atmosphere of N₂ at 80° C. on a 0.2-1.0 mmol scale (unless indicated otherwise) using a primary amine (Conditions A) or a secondary amine (Conditions B) in anhydrous PhMe (0.4 M) for a duration of 18-48 h. [b] Yield was determined by ¹H NMR spectroscopy of the crude reaction mixture, using mesitylene as an internal standard. [c] Isolated yield on a 1.0 mmol scale. [d] Carried out using 2.0 equiv amine instead. [e] Reactions were carried out using n-butylamine (conditions A) or piperidine (conditions B) and subsequently quenched with 1N aq. HCl. Yields reflect isolated yields. N.D.=Not Detected. [f] Carried out using 5 equiv PMHS instead. [g] Carried out using 4 equiv n-butylamine and 6 equiv DMMS instead. [h] Carried out on a 0.5-1 mmol scale using conditions A without acidic workup, followed by NaBH₄ (1.5-3 equiv) reduction at 65° C. for 4-9 h. [i] Carried out using conditions B instead, followed by NaBH₄ (3 equiv) reduction at 65° C. for 9 h. [j] Carried out using 3 equiv n-butylamine and 6 equiv DMMS instead.

With optimized conditions defined, the single-step catalytic semi-reductive imination (3) and enamination (4) of esters was investigated, a direct functional group interconversion. Aryl esters were directly converted to imines using various primary amines (3a-3c), while a cinnamate was transformed into hydrazone 3d when phenyhydrazine was employed as the nucleophile. The enamination of aliphatic esters could be carried out as well when using an assortment of cyclic amines (4a-4d). Notably, when diethylamine was employed instead, enamine 4e was formed in moderate yield. Thiophene, morpholine, and sulfide functionality were all tolerated under these reaction conditions (4f-4h).

The ZrH-catalyzed semi-reduction of esters to aldehydes was explored next through the implementation of a hydrolytic workup. Methyl and ethyl benzoates (1d, 1e) smoothly reacted to afford benzaldehydes in high yields, whereas more sterically encumbered esters either resulted in lower conversion of the starting material (1f) or amidation (1 g). The partial reduction of 1e proved to be scalable, furnishing greater than 1 gram of aldehyde 6 on a 10 mmol scale. In general, various esters bearing ether, halide, N-heterocyclic, and sulfide functionality were amenable to the catalytic semi-reduction (7-11). Additionally, reduction of methyl (E)-2-methyl-3-phenylacrylate provided enal 12 in 71% yield, extending the utility of this protocol to the preparation of α,β-unsaturated aldehydes. Finally, aliphatic aldehydes 13 and 14 were obtained in moderate yields.

At elevated temperatures competitive nitrile reduction becomes problematic under this catalytic manifold. For example, substrate 1l underwent unselective reduction to afford terephthaladehyde. In accord with prior observations regarding the steric sensitivity of this catalytic system, esters bearing an α-quaternary or tertiary carbons (e.g. 1m and 2k) exhibit poor reactivity.

The use of an amine to interrupt traditional metal hydride-mediated ester reduction enables concise entry to valuable reactive intermediates that can be directly telescoped through multi-step synthetic sequences, necessitating only a single purification step. For example, the interception of imine intermediates with nucleophiles delivers access to α-alkylated secondary amines (FIG. 4B, 15 and 16). Alternatively, the ester starting material can instead serve as the nucleophilic component through generation of an enamine. This latter strategy was displayed through the single-flask multicomponent synthesis of α-alkylated aldehyde 17 in 59% yield when benzyl bromide was added after enamine formation (FIG. 4B).

The potential of this catalytic manifold for the two-stage single-flask reductive amination of esters when incorporating a NaBH₄ reduction prior to workup was illustrated. An assortment of benzylic, allylic, and aliphatic amines were isolated in 60-72% yield (FIG. 4B, 18-23). The juxtaposition of imine 3a, aldehyde 5, and amine 18 best exemplify the controllable selectivity attainable through this catalytic manifold.

### Characterization Data for Imine, Hydrazone, and Enamine Products (FIG. 4A)

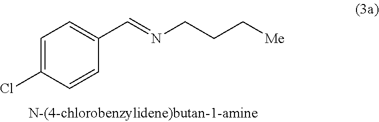 [SMILES: CCCC/N=C/c1ccc(Cl)cc1]

Following general procedure A using methyl 4-chlorobenzoate (1a, 170.6 mg, 1.0 mmol), the reaction was carried out for 21 h. In lieu of an acidic workup, the crude residue was directly concentrated and a ¹H NMR yield was obtained using mesitylene (13.9 μL, 0.10 mmol) as an internal standard (91% ¹H NMR yield).

The spectroscopic data for this compound match those previously reported in the literature.

Diagnostic signals identified in the ¹H NMR of the crude residue:

¹H NMR (500 MHz, CDCl₃) δ 8.22 (s, 1H), 7.69-7.64 (m, 2H), 7.39-7.36 (m, 2H), 3.62 (td, J=7.0, 1.4 Hz, 2H), 1.74-1.66 (m, 2H), 1.45-1.37 (m, 2H), 0.97 (t, J=7.4 Hz, 3H).

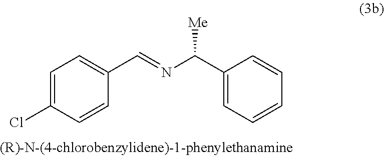 [SMILES: C[C@@H](/N=C/c1ccc(Cl)cc1)c1ccccc1]

Following general procedure A using methyl 4-chlorobenzoate (34.1 mg, 0.20 mmol) and (R)-α-methylbenzylamine (43.5 μL, 0.34 mmol, 1.7 equiv), the reaction was carried out for 22 h. In lieu of an acidic workup, the crude residue was directly concentrated and a ¹H NMR yield was obtained using mesitylene (13.9 μL, 0.10 mmol) as an internal standard (60% ¹H NMR yield).

The spectroscopic data for this compound match those previously reported in the literature.

Diagnostic signals identified in the ¹H NMR of the crude residue:

¹H NMR (600 MHZ, CDCl₃) δ 8.32 (s, 1H), 7.73-7.71 (m, 2H), 7.44-7.41 (m, 2H), 4.54 (q, J=6.6 Hz, 1H), 1.59 (d, J=6.6 Hz, 3H).

The formation of 4-chloro-N-(1-phenylethyl)benzamide was also apparent.

Diagnostic signals identified in the ¹H NMR of the crude residue:

¹H NMR (600 MHZ, CDCl₃) δ 6.37 (br s, 1H), 5.33 (p, J=7.1 Hz, 1H), 1.61 (d, J=6.9 Hz, 3H).

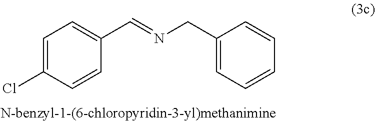 [SMILES: Clc1ccc(/C=N/Cc2ccccc2)cc1]

Following general procedure A using methyl 6-chloronicotinate (34.3 mg, 0.20 mmol) and benzylamine (37.1 μL), the reaction was carried out for 18 h. In lieu of an acidic workup, the crude residue was directly concentrated and a ¹H NMR yield was obtained using mesitylene (13.9 μL, 0.10 mmol) as an internal standard (64% ¹H NMR yield).

The spectroscopic data for this compound match those previously reported in the literature.

Diagnostic Signals Identified in the ¹H NMR of the Crude Residue (Excluding Signals Overlapping with Residual PhMe):

¹H NMR (600 MHZ, CDCl₃) δ 8.64 (d, J=2.3 Hz, 1H), 8.37 (s, 1H), 8.15 (dd, J=8.3, 2.4 Hz, 1H), 4.85 (s, 2H).

The formation of N-benzyl-6-chloronicotinamide was also apparent.

### Diagnostic Signals Identified in the ¹H NMR of the Crude Residue:

¹H NMR (600 MHZ, CDCl₃) δ 8.77 (d, J=2.6 Hz, 1H), 8.08 (dd, J=8.3, 2.5 Hz, 1H), 4.63 (d, J=5.7 Hz, 2H).

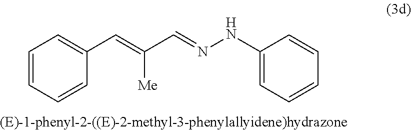 [SMILES: CC(/C=N/Nc1ccccc1)=C\c1ccccc1]

Following general procedure A using methyl 3-methyl-3-phenylacrylate (35.2 mg, 0.20 mmol) and phenylhydrazine (36.8 mg, 0.34 mmol, 1.7 equiv), the reaction was carried out for 20 h. In lieu of an acidic workup, the crude residue was directly concentrated and a 1H NMR yield was obtained using mesitylene (13.9 μL, 0.10 mmol) as an internal standard (74% ¹H NMR yield).

The spectroscopic data for this compound match those previously reported in the literature.

Diagnostic Signals Identified in the ¹H NMR of the Crude Residue (Excluding Signals Overlapping with Residual PhMe):

¹H NMR (500 MHZ, CDCl₃) δ 7.56 (br s, 1H), 7.44 (s, 1H), 7.41-7.36 (m, 4H), 7.06 (d, J=7.5 Hz, 2H), 6.86 (t, J=7.3 Hz, 1H), 6.55 (s, 1H), 2.25 (d, J=1.3 Hz, 3H).

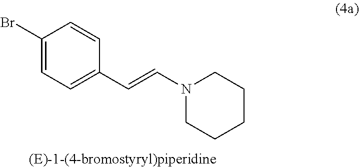 [SMILES: Brc1ccc(/C=C/N2CCCCC2)cc1]

0.2 mmol scale, crude ¹H NMR analysis:

Following general procedure B using methyl 2-(4-bromophenyl)acetate (45.8 mg, 0.2 mmol) the reaction was carried out for 23 h. In lieu of an acidic workup, the crude residue was directly concentrated and a ¹H NMR yield was obtained using mesitylene (13.9 μL, 0.10 mmol) as an internal standard (≥99% ¹H NMR yield).

The spectroscopic data for this compound match those previously reported in the literature.

### Diagnostic Signals Identified in the ¹H NMR of the Crude Residue:

¹H NMR (600 MHZ, CDCl₃) δ 7.31-7.27 (m, 2H), 7.05-7.01 (m, 2H), 6.65 (d, J=14.0 Hz, 1H), 5.26 (d, J=14.0 Hz, 1H), 3.05-3.01 (m, 4H), 1.64-1.56 (m, 6H).

1.0 mmol scale, isolated yield of 4a:

Following general procedure B using methyl 2-(4-bromophenyl)acetate (229.1 mg, 1.0 mmol) the reaction was carried out for 22 h. In lieu of an acidic workup, the crude residue was directly concentrated and a ¹H NMR yield was obtained using mesitylene (27.8 μL, 0.20 mmol) as an internal standard (≥99% ¹H NMR yield). The crude residue was purified via flash column chromatography using activated aluminum oxide (neutral, Brockmann Grade I, 58 Å) (elutes using 1% NEt₃ in Hex→5% EtOAc/1% NEt₃ in Hex). The title compound was obtained as a white solid (179.6 mg, 67% yield).

The spectroscopic data for this compound match those previously reported in the literature.

¹H NMR (600 MHZ, CDCl₃) δ 7.29 (d, J=8.3 Hz, 2H), 7.03 (d, J=8.5 Hz, 2H), 6.65 (d, J=14.0 Hz, 1H), 5.26 (d, J=14.0 Hz, 1H), 3.07-3.00 (m, 4H), 1.67-1.54 (m, 6H).

¹³C NMR (151 MHZ, CDCl₃) δ 140.8, 138.8, 131.5, 125.3, 116.5, 97.9, 49.7, 25.4, 24.4.

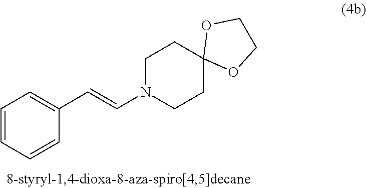 [SMILES: C(=C\N1CCC2(CC1)OCCO2)/c1ccccc1]

Following general procedure B using methyl phenylacetate (28.2 μL, 0.20 mmol) and 1,4-dioxa-8-azaspiro[4.5]decane (51.3 μL, 0.40 mmol, 2.0 equiv), the reaction was carried out for 23 h. In lieu of an acidic workup, the crude residue was directly concentrated and a ¹H NMR yield was obtained using mesitylene (13.9 μL, 0.10 mmol) as an internal standard (76% ¹H NMR yield).

The spectroscopic data for this compound match those previously reported in the literature.

Diagnostic Signals Identified in the ¹H NMR of the Crude Residue (Excluding Signals Overlapping with Residual PhMe):

¹H NMR (500 MHZ, CDCl₃) δ 7.01 (tt, J=6.8, 1.8 Hz, 1H), 6.66 (d, J=14.1 Hz, 1H), 5.39 (d, J=14.1 Hz, 1H), 3.98 (s, 4H), 3.22-3.16 (m, 4H), 1.80-1.76 (m, 4H).

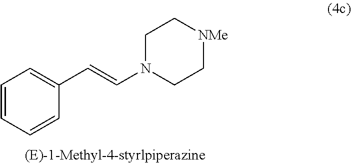 [SMILES: CN1CCN(/C=C/c2ccccc2)CC1]

Following general procedure B using methyl phenylacetate (28.2 μL, 0.20 mmol) and N-methylpiperazine (33.3 μL, 0.30 mmol, 1.5 equiv), the reaction was carried out for 24 h. In lieu of an acidic workup, the crude residue was directly concentrated and a ¹H NMR yield was obtained using mesitylene (13.9 μL, 0.10 mmol) as an internal standard (47% ¹H NMR yield).

The spectroscopic data for this compound match those previously reported in the literature.

Diagnostic Signals Identified in the ¹H NMR of the Crude Residue (Excluding Signals Overlapping with Residual PhMe):

¹H NMR (400 MHZ, CDCl₃) δ 7.05-7.00 (m, 1H), 6.66 (d, J=14.1 Hz, 1H), 5.41 (d, J=14.1 Hz, 1H), 3.11-3.06 (m, 4H), 2.50-2.46 (m, 4H), 2.34 (s, 3H).

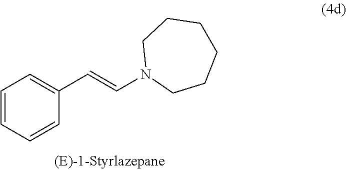 [SMILES: C(=C\N1CCCCCC1)/c1ccccc1]

Following general procedure B using methyl phenylacetate (28.2 μL, 0.20 mmol) and azepane (33.8 μL, 0.30 mmol, 1.5 equiv), the reaction was carried out for 21 h. In lieu of an acidic workup, the crude residue was directly concentrated and a ¹H NMR yield was obtained using mesitylene (13.9 μL, 0.10 mmol) as an internal standard (≥99% ¹H NMR yield).

The spectroscopic data for this compound match those previously reported in the literature.

### Diagnostic Signals Identified in the ¹H NMR of the Crude Residue:

H NMR (600 MHZ, CDCl₃) δ 7.21-7.13 (m, 4H), 6.92 (tt, J=6.9, 1.6 Hz, 1H), 6.85 (d, J=13.8 Hz, 1H), 5.09 (d, J=13.8 Hz, 1H), 3.29-3.24 (m, 4H), 1.76-1.70 (m, 4H, overlapping), 1.59-1.57 (m, 4H).

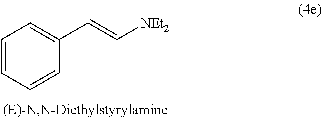

0.2 mmol scale, crude ¹H NMR analysis:

Following general procedure B using methyl phenylacetate (28.2 μL, 0.20 mmol) and diethylamine (31.0 μL, 0.30 mmol, 1.5 equiv), the reaction was carried out for 21 h. In lieu of an acidic workup, the crude residue was directly concentrated and a ¹H NMR yield was obtained using mesitylene (13.9 μL, 0.10 mmol) as an internal standard (41% ¹H NMR yield).

The spectroscopic data for this compound match those previously reported in the literature.

Diagnostic Signals Identified in the ¹H NMR of the Crude Residue (Excluding Signals Overlapping with Residual PhMe):

¹H NMR (500 MHZ, CDCl₃) δ 6.94 (tt, J=7.0, 1.6 Hz, 1H), 6.76 (d, J=14.0 Hz, 1H), 5.17 (d, J=14.0 Hz, 1H), 3.16 (q, J=7.1 Hz, 4H), 1.15 (t, J=7.1 Hz, 6H).

1.0 mmol scale, isolated yield of 4e:

Following general procedure B using methyl phenylacetate (141.0 μL, 1.0 mmol) the reaction was carried out for 22 h. In lieu of an acidic workup, the crude residue was directly purified via flash column chromatography using activated aluminum oxide (neutral, Brockmann Grade I, 58 Å) (elutes using 10% EtOAc/1% NEt₃ in Hex). The title compound was obtained as a colorless oil (69 mg, 39% yield).

¹H NMR (600 MHZ, CDCl₃) δ 7.20-7.18 (m, 2H), 7.16-7.14 (m, 2H), 6.94 (tt, J=7.2, 1.5 Hz, 1H), 6.76 (d, J=14.0 Hz, 1H), 5.17 (d, J=14.0 Hz, 1H), 3.16 (q, J=7.1 Hz, 4H), 1.16 (t, J=7.1 Hz, 6H).

¹³C NMR (151 MHZ, CDCl₃) δ 140.4, 137.8, 128.6, 123.3, 122.9, 96.2, 45.4, 13.3.

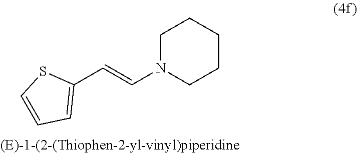 [SMILES: C(=C\N1CCCCC1)/c1cccs1]

Following general procedure B using methyl 2-thienylacetate (31.2 mg, 0.2 mmol), the reaction was carried out for 20 h. In lieu of an acidic workup, the crude residue was directly concentrated and a ¹H NMR yield was obtained using mesitylene (13.9 μL, 0.10 mmol) as an internal standard (97% ¹H NMR yield).

The spectroscopic data for this compound match those previously reported in the literature.

### Diagnostic Signals Identified in the ¹H NMR of the Crude Residue:

¹H NMR (500 MHZ, CDCl₃) δ 6.88-6.84 (m, 2H), 6.64 (dd, J=3.3, 1.4 Hz, 1H), 6.57 (d, J=13.9 Hz, 1H), 5.54 (d, J=13.9 Hz, 1H), 3.03-2.97 (m, 4H), 1.65-1.54 (m, 6H).

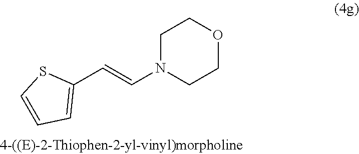 [SMILES: C(=C\N1CCOCC1)/c1cccs1]

Following general procedure B using methyl 2-thienylacetate (31.2 mg, 0.20 mmol) and morpholine (25.9 μL, 0.30 mmol, 1.5 equiv), the reaction was carried out for 20 h. In lieu of an acidic workup, the crude residue was directly concentrated and a ¹H NMR yield was obtained using mesitylene (13.9 μL, 0.10 mmol) as an internal standard (88% ¹H NMR yield).

The spectroscopic data for this compound match those previously reported in the literature.

### Diagnostic Signals Identified in the ¹H NMR of the Crude Residue:

¹H NMR (500 MHZ, CDCl₃) δ 6.92-6.87 (m, 2H), 6.69 (d, J=3.3 Hz, 1H), 6.53 (d, J=14.0 Hz, 1H), 5.61 (d, J=13.9 Hz, 1H), 3.78-3.74 (m, 4H), 3.03-2.97 (m, 4H).

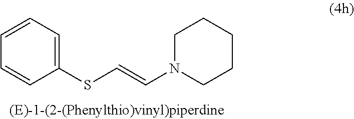 [SMILES: C(=C\N1CCCCC1)/Sc1ccccc1]

Following general procedure B using methyl 2-(phenylthiol)acetate (36.5 mg, 0.2 mmol), the reaction was carried out for 18 h. In lieu of an acidic workup, the crude residue was directly concentrated and a ¹H NMR yield was obtained using mesitylene (13.9 μL, 0.10 mmol) as an internal standard (97% ¹H NMR yield).

The spectroscopic data for this compound match those previously reported in the literature.

Diagnostic Signals Identified in the ¹H NMR of the Crude Residue (Excluding Signals Overlapping with Residual PhMe):

¹H NMR (600 MHZ, CDCl₃) δ 7.07 (tt, J=7.4, 1.4 Hz, 1H), 6.81 (dt, J=1.4, 0.7 Hz, 2H), 6.54 (d, J=12.9 Hz, 1H), 4.78 (d, J=12.9 Hz, 1H), 3.07-3.03 (m, 4H), 1.62-1.58 (m, 6H).

### Characterization Data for Aldehyde Products (FIG. 4A)

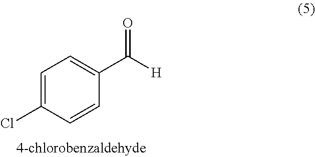

Following general procedure A using methyl 4-chlorobenzoate (1a) (170.6 mg, 1.0 mmol), the reaction was carried for 23 h. The crude residue was then purified with the aid of CombiFlash Nextgen 300 (12 g gold cartridge, elutes using 2.5% Et₂O in Pentane 30 mL/min). The title compound was obtained as a white solid (107.6 mg, 76% yield).

The spectroscopic data for this compound match those previously reported in the literature.

¹H NMR (400 MHZ, CDCl₃) δ 9.99 (s, 1H), 7.85-7.80 (m, 2H), 7.54-7.50 (m, 2H).

¹³C NMR (101 MHZ, CDCl₃) δ 190.1, 141.1, 134.9, 131.1, 129.6.

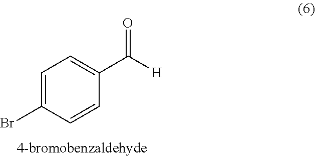

(Using 1d): Following general procedure A using methyl 4-bromobenzoate (1d) (215.05 mg, 1.0 mmol), the reaction was carried out for 21 h. A ¹H NMR yield was obtained using mesitylene as an internal standard (79% ¹H NMR yield).

(Using 1e): Following general procedure A using ethyl 4-bromobenzoate (1e) (163.6 μL, 1.0 mmol), the reaction was carried out for 21.5 h. The crude residue was then purified with the aid of CombiFlash Nextgen 300 (12 g gold cartridge, elutes using 5% Et₂O in Pentane at 30 mL/min). To remove residual silicon-based impurities, the title compound was further purified by a second round of chromatography (12 g gold cartridge, elutes using 5% Et₂O in Pentane at 30 mL/min). The title compound was obtained as a crystalline white solid (138.5 mg, 75% yield).

The spectroscopic data for this compound match those previously reported in the literature.

¹H NMR (400 MHZ, CDCl₃) δ 9.98 (s, 1H), 7.78-7.73 (m, 2H), 7.72-7.67 (m, 2H).

¹³C NMR (101 MHZ, CDCl₃) δ 191.2, 135.2, 132.6, 131.1, 129.9.

(Using 1e, 10.0 mmol): Following general procedure A using ethyl 4-bromobenzoate (1e) (1.65 mL, 10.0 mmol), the reaction was carried out for 48 h. The crude residue was then purified with the aid of CombiFlash Nextgen 300 (100 gold cartridge, elutes using 2.5% Et₂O in Pentane at 30 mL/min). The title compound was obtained as a white solid (1.13 g, 61% yield).

(Using 1f): Following general procedure A using isopropyl 4-bromobenzoate (1f) (138.6 mg, 0.5 mmol), the reaction was carried out for 23 h. The crude residue was then purified with the aid of CombiFlash Nextgen 300 (12 g gold cartridge, elutes using 2.5% Et₂O in Hexane at 30 mL/min). The title compound was obtained as a white solid (38.9 mg, 42% yield).

(Using 1 g): Following general procedure A using phenyl 4-bromobenzoate (1 g) (138.6 mg, 0.5 mmol), the reaction was carried out for 23 h. Crude ¹H NMR analysis showed full conversion of 1h to 4-bromo-N-butylbenzamide.

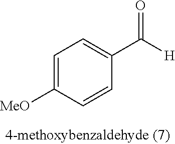

Following general procedure A using methyl 4-methoxybenzoate (166.2 mg, 1.0 mmol), the reaction was carried out for 20 h. The crude residue was then purified with the aid of CombiFlash Nextgen 300 (12 g gold cartridge, elutes using 100% Et₂O at 30 mL/min). To remove residual silicon-based impurities, the title compound was further purified through a plug of neutral Brockmann grade alumina (approx. 2.0 g alumina, elutes using 100% Hex→50% Et₂O/Hex). The title compound was obtained as a yellow oil (98.0 mg, 72% yield).

The spectroscopic data for this compound match those previously reported in the literature.

¹H NMR (500 MHZ, CDCl₃) δ 9.89 (s, 1H), 7.84 (d, J=7.9 Hz, 2H), 7.00 (d, J=8.4 Hz, 2H), 3.89 (s, 3H).

¹³C NMR (101 MHZ, CDCl₃) δ 190.9, 164.7, 132.1, 130.1, 114.4, 55.7.

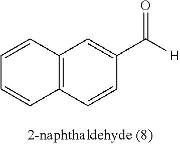 [SMILES: O=Cc1ccc2ccccc2c1]

Following general procedure A using methyl 2-naphthoate (186.2 mg, 1.0 mmol), the reaction was carried out for 22.5 h. The crude residue was then purified with the aid of CombiFlash Nextgen 300 (12 g gold cartridge, elutes using 100% Hexanes at 30 mL/min). The title compound was obtained as a white solid (131.0 mg, 84% yield).

The spectroscopic data for this compound match those previously reported in the literature.

¹H NMR (600 MHZ, CDCl₃) δ 10.15 (s, 1H), 8.31 (s, 1H), 7.99 (d, J=8.1 Hz, 1H), 7.95 (m, 1H), 7.93-7.87 (m, 2H), 7.64 (ddd, J=8.2, 6.9, 1.3 Hz, 1H), 7.58 (ddd, J=8.2, 6.9, 1.3 Hz, 1H).

¹³C NMR (101 MHZ, CDCl₃) δ 192.3, 136.4, 134.6, 134.1, 132.6, 129.5, 129.1, 129.1, 128.1, 127.1, 122.7.

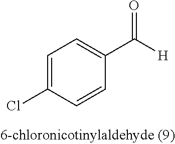

Following general procedure A using methyl 6-chloronicotinate (171.6 mg, 1.0 mmol), the reaction was carried out for 22 h. The crude residue was then purified with the aid of CombiFlash Nextgen 300 (12 g gold cartridge, elutes using 5% Et₂O in Pentane at 30 mL/min). The title compound was obtained as a white solid (83.1 mg, 59% yield).

The spectroscopic data for this compound match those previously reported in the literature.

¹H NMR (600 MHZ, CDCl₃) δ 10.10 (s, 1H), 8.87 (dd, J=2.4, 0.7 Hz, 1H), 8.14 (dd, J=8.2, 2.3 Hz, 1H), 7.52 (dt, J=8.2, 0.7 Hz, 1H).

¹³C NMR (101 MHZ, CDCl₃) δ 189.3, 157.0, 152.5, 138.1, 130.5, 125.3.

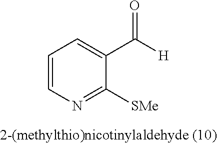

Following general procedure A using methyl 2-(methylthio) nicotinate (171.6 mg, 1.0 mmol), the reaction was carried out for 22 h. The crude residue was then purified with the aid of CombiFlash Nextgen 300 (12 g gold cartridge, elutes using 5% Et₂O in Hexane at 30 mL/min). The title compound was obtained as a white solid (93.7 mg, 61% yield).

The ¹H NMR spectroscopic data for this compound match a previous literature report.

¹H NMR (500 MHZ, CDCl₃) δ 10.18 (dd, J=7.0, 3.4 Hz, 1H), 8.60-8.54 (m, 1H), 7.99-7.93 (m, 1H), 7.13 (dtd, J=7.7, 4.8, 2.5 Hz, 1H), 2.56 (dd, J=6.4, 3.1 Hz, 3H).

¹³C NMR (101 MHZ, CDCl₃) δ 190.1, 162.8, 153.6, 139.8, 128.5, 118.8, 13.1.

IR (neat): 2746, 1682, 1541, 1377, 1138, 1095, 1061, 801, 684 cm⁻¹.

HRMS (+ESI): Calculated for C₇H₇SNO [M+H]⁺: 154.0321. Found: 154.0321.

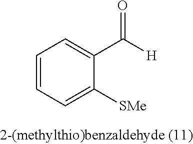

Following general procedure A using ethyl 2-thiomethylbenzoate (196.3 mg, 1.0 mmol), the reaction was carried out for 21.5 h. The crude residue was then purified with the aid of CombiFlash Nextgen 300 (12 g gold cartridge, elutes using 5% Et₂O in Pentane at 30 mL/min). To remove residual silicon-based impurities, the title compound was further purified by a second round of chromatography (12 g gold cartridge, elutes using 5% Et₂O in Pentane at 30 mL/min). The title compound was obtained as a yellow oil (120.2 mg, 79% yield).

The spectroscopic data for this compound match those previously reported in the literature.

¹H NMR (600 MHZ, CDCl₃) δ 10.26 (s, 1H), 7.80 (dd, J=7.7, 1.3 Hz, 1H), 7.53 (tdd, J=7.3, 1.6, 0.6 Hz, 1H), 7.33 (d, J=8.0 Hz, 1H), 7.28 (t, J=7.5 Hz, 1H), 2.49 (s, 3H).

¹³C NMR (101 MHZ, CDCl₃) δ 191.5, 143.5, 134.1, 133.5, 133.0, 125.5, 124.5, 15.6.

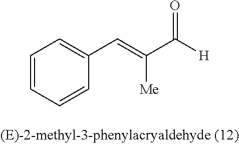 [SMILES: C/C(C=O)=C\c1ccccc1]

Following general procedure A using (E)-methyl-2-methyl-3-phenylacrylate (176.2 mg, 1.0 mmol), the reaction was carried out for 19 h. The crude residue was then purified with the aid of CombiFlash Nextgen 300 (12 g gold cartridge, elutes using 5% Et₂O in Pentane at 30 mL/min). The title compound was obtained as a pale yellow oil (104.1 mg, 71% yield).

The spectroscopic data for this compound match those previously reported in the literature.

¹H NMR (400 MHZ, CDCl₃) δ 9.60 (s, 1H), 7.57-7.50 (m, 2H), 7.49-7.43 (m, 2H), 7.43-7.37 (m, 1H), 7.28 (br s, 1H), 2.09 (d, J=1.4 Hz, 3H).

¹³C NMR (101 MHZ, CDCl₃) δ 195.7, 150.0, 138.5, 135.3, 130.2, 129.7, 128.9, 11.1.

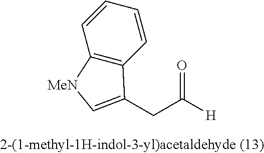 [SMILES: Cn1cc(CC=O)c2ccccc21]

Following general procedure B using methyl N-methylindol-3-acetate (99.6 mg, 0.49 mmol) and PMHS (150 μL, 2.5 mmol), the reaction was carried out for 20 h. The crude residue was purified with the aid of CombiFlash Nextgen 300 (12 g gold cartridge, elutes using 15% Et₂O in Pentane at 30 mL/min). The title compound was obtained as a viscous, pale yellow oil (48.9 mg, 58% yield).

The spectroscopic data for this compound match those previously reported in the literature.

¹H NMR (400 MHZ, CDCl₃) δ 9.74 (t, J=2.5 Hz, 1H), 7.53 (dt, J=7.9, 1.0 Hz, 1H), 7.35-7.30 (m, 1H), 7.26 (ddd, J=7.9, 6.8, 1.1 Hz, 1H), 7.14 (ddd, J=8.0, 6.9, 1.1, 1H), 7.00 (s, 1H), 3.79-3.78 (m, 2H), 3.77 (s, 3H).

¹³C NMR (101 MHZ, CDCl₃) δ 199.6, 137.2, 128.2, 128.0, 122.2. 119.6, 118.7, 109.6, 104.4, 40.4, 32.9.

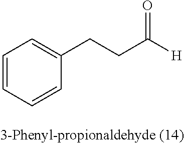

In a nitrogen-filled glovebox Cp₂ZrCl₂ (29.2 mg, 10.0 mol %) was weighed into a flame dried 20×125 mm reaction tube equipped with a magnetic stir bar. Anhydrous PhMe (2.5 mL, 0.4 M) was injected into the reaction tube, followed by the injection of methyl 3-phenylpropionate (164.2 mg, 1.0 mmol), DMMS (733 μL, 6.0 mmol, 6.0 equiv), and n-butylamine (395 μL, 4.0 mmol, 4.0 equiv). The mixture was stirred for 3 minutes, then the tube was capped with an open top screw cap equipped with a Teflon-lined silicon septum and removed from the glovebox. The tube was connected to a N₂ flow and left to stir at 80° C. for 20 h. The reaction mixture was then quenched with 10 mL of 4 M HCl, diluted with 20 mL of H₂O, and stirred at room temperature. The solution was diluted with ca. 10 mL of brine and the aqueous layer was extracted with CH₂Cl₂ (ca. 3×10 mL). The combined organic washes were dried over Na₂SO₄, vacuum filtered, and concentrated in vacuo with the aid of a rotary evaporator. The crude residue was purified via flash column chromatography (elutes using 5% EtOAc in CH₂Cl₂). The title compound was obtained as a colorless oil (68.6 mg, 51% yield).

The spectroscopic data for this compound match those previously reported in the literature.

¹H NMR (600 MHZ, CDCl₃) δ 9.83 (t, J=1.4 Hz, 1H), 7.31-7.29 (m, 2H), 7.22-7.19 (m, 3H), 2.97 (t, J=7.6 Hz, 2H), 2.79 (td, J=7.6 Hz, 1.3 Hz, 2H).

¹³C NMR (151 MHZ, CDCl₃) δ 201.7, 140.4, 128.7, 128.4, 126.4, 45.4, 28.2.

### Characterization Data for Amines 15 & 16 (FIG. 4B)

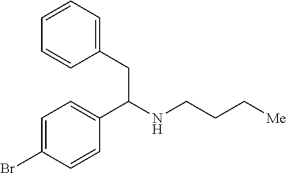 [SMILES: CCCCNC(Cc1ccccc1)c1ccc(Br)cc1]

15: Cp₂ZrCl₂ (14.6 mg, 5.0 mol %) was weighed into a flame-dried 20×125 mm reaction tube equipped with a magnetic stir bar. The reaction tube was capped with an open top screw cap equipped with a Teflon-lined silicon septum and sealed with electrical tape. The reaction tube was then evacuated and backfilled with nitrogen (this process was repeated to a total of three times). The catalyst was dissolved in 2.5 mL of anhydrous PhMe (0.4 M). Ethyl 4-bromo benzoate (163.3 μL, 1.0 mmol) was injected via microsyringe. DEMS (480.6 μL, 3.0 equiv) was injected into the reaction mixture, followed by the injection of n-butylamine (168.0 μL, 1.7 equiv) using a Hamilton gastight glass microsyringe. The septum of the reaction tube was sealed with wax and the reaction solution was stirred at 500 rpm at 80° C. After 21 h, the reaction solution was cooled to 0° C. and diluted with 3.5 mL of anhydrous THF. Benzylmagnesium chloride (2.0 mL [2.0 M solution in THF], 4.0 equiv) was injected dropwise over approx. 5 min. After 40 min, the reaction mixture was warmed to 23° C. After 5 h, the reaction solution was quenched with 5 mL of 1 M HCl. The aqueous layer was washed three times with ca. 10 mL Et₂O. The combined organic layers were dried over MgSO₄, vacuum filtered, and concentrated in vacuo with the aid of a rotary evaporator. The crude residue was then purified with the aid of CombiFlash Nextgen 300 (10 g cartridge, elutes using 5% EtOAc/1% NEt₃/Hex at 30 mL/min). The desired product 15 was obtained as a pale yellow oil (254.7 mg, 94.3% purity, 72% yield).

¹H NMR (600 MHZ, CDCl₃) δ 7.45-7.40 (m, 2H), 7.28-7.24 (m, 2H), 7.23-7.16 (m, 3H), 7.10 (d, J=6.8 Hz, 2H), 3.81 (t, J=7.1 Hz, 1H), 2.88 (d, J=5.8 Hz, 2H), 2.41-2.29 (m, 2H), 1.34 (d, J=7.8 Hz, 2H), 1.19 (dpd, J=14.4, 7.3, 2.2 Hz, 2H), 0.81 (t, J=7.3 Hz, 3H).

¹³C NMR (101 MHZ, CDCl₃) δ 143.3, 138.6, 131.5, 129.4, 129.2, 128.6, 126.6, 120.7, 64.5, 47.6, 45.4, 32.3, 20.4, 14.1.

IR (neat): 3027, 2925, 1602, 1484, 1454, 1117, 1070, 1009, 818, 745, 697, 555 cm⁻¹.

LRMS (LC-MS): Calculated for C₂₂H₂₃BrN [M]: 332.28. Found: 332.27.

HRMS (+ESI): Calculated for C₂₂H₂₃BrN [M+H]⁺: 332.1008, 334.0988. Found: 332.0981, 334.0959.

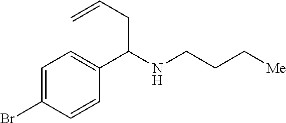 [SMILES: C=CCC(NCCCC)c1ccc(Br)cc1]

16: Cp₂ZrCl₂ (14.6 mg, 5.0 mol %) was weighed into a flame-dried 20×125 mm reaction tube equipped with a magnetic stir bar. The reaction tube was capped with an open top screw cap equipped with a Teflon-lined silicon septum and sealed with electrical tape. The reaction tube was then evacuated and backfilled with nitrogen (this process was repeated to a total of three times). The catalyst was dissolved in 2.5 mL of anhydrous PhMe (0.4 M). Ethyl 4-bromo benzoate (163.3 μL, 1.0 mmol) was injected via microsyringe. DEMS (480.6 μL, 3.0 equiv) was injected into the reaction mixture, followed by the injection of n-butylamine (168.0 μL, 1.7 equiv) using a Hamilton gastight glass microsyringe. The septum of the reaction tube was sealed with wax and the reaction solution was stirred at 500 rpm at 80° C. After 19.5 h, the reaction solution was cooled to 0° C. and diluted with 3.5 mL of anhydrous THF. Allylmagnesium bromide (4.0 mL [1.0 M solution in Et₂O], 4.0 equiv) was injected dropwise over approx. 5 min. After 45 min, the reaction mixture was warmed to 23° C. After 5 h, the reaction solution was quenched with 5 mL of 1 M HCl. The aqueous layer was washed three times with ca. 10 mL Et₂O. The combined organic layers were dried over MgSO₄, vacuum filtered, and concentrated in vacuo with the aid of a rotary evaporator. The crude residue was then purified with the aid of CombiFlash Nextgen 300 (10 g cartridge, elutes using 5% EtOAc/1% NEt₃/Hex at 30 mL/min). The desired product 16 was obtained as a pale yellow oil (161.6 mg, 57% yield).

¹H NMR (400 MHZ, CDCl₃) δ 7.47 (m, 2H), 7.22-7.16 (m, 2H), 5.69 (dddd, J=16.7, 10.2, 7.9, 6.3 Hz, 1H), 5.11-5.01 (m, 2H), 3.60 (dd, J=7.6, 5.9 Hz, 1H), 2.43-2.26 (m, 4H), 1.41 (p, J=6.8 Hz, 3H), 1.28 (dpd, J=14.2, 7.2, 2.0 Hz, 2H), 0.86 (t, J=7.3 Hz, 3H).

¹³C NMR (101 MHZ, CDCl₃) δ 143.5, 135.3, 131.5, 129.1, 120.6, 118.0, 62.3, 47.6, 43.2, 32.4, 20.6, 14.1.

IR (neat): 3077, 2956, 2926, 1639, 1485, 1464, 1009, 916, 820 cm⁻¹.

HRMS (+ESI): Calculated for C₁₄H₂₀BrN [M+H]⁺: 282.0852, 284.0831. Found: 282.0849, 284.0829.

### Characterization Data for Aldehyde 17 (FIG. 4B)

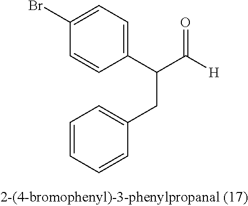 [SMILES: O=CC(Cc1ccccc1)c1ccc(Br)cc1]

The title compound was obtained with slight procedural modification to general procedure B. PhMe (2.5 mL, 0.4 M), piperidine (148.1 μL, 1.5 equiv), methyl 2-(4-bromophenyl)acetate (2a) (229.0 mg, 1.0 mmol), and DEMS (480.6 μL, 3.0 equiv) were stirred at 80° C. for 23 h. Then, the reaction was cooled to 23° C. and benzyl bromide (0.48 mL, 4.0 equiv) was injected dropwise into the reaction tube. The reaction solution was re-heated to 80° C. for 24 hours. Following acidic workup, the crude residue was purified with the aid of CombiFlash Nextgen 300 (12 g gold cartridge, elutes using 5% Et₂O in Hexanes at 30 mL/min). The title compound was obtained as an orange oil (170.2 mg, 59% yield).

¹H NMR (600 MHZ, CDCl₃) δ 9.73 (d, J=1.4 Hz, 1H), 7.49-7.44 (m, 2H), 7.25-7.20 (m, 2H), 7.19-7.15 (m, 1H), 7.06-7.02 (m, 2H), 7.01-6.98 (m, 2H), 3.81 (ddd, J=8.1, 6.4, 1.4 Hz, 1H), 3.45 (dd, J=14.0, 6.4 Hz, 1H), 2.94 (dd, J=14.0, 8.3 Hz, 1H).

¹³C NMR (151 MHZ, CDCl₃) δ 199.4, 138.4, 134.8, 132.3, 130.8, 128.6, 129.1, 126.6, 122.0, 60.4, 36.3.

IR (neat): 3027, 2924, 1721, 1487, 1072, 1010, 698, 523 cm⁻¹.

HRMS (−ESI): Calculated for C₁₅H₁₃BrO [M+H]: 289.0223, 291.0202. Found: 289.0058, 291.0202.

### Characterization Data for Amines 18-23 (FIG. 4B)

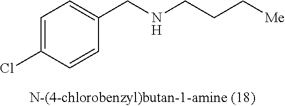 [SMILES: CCCCNCc1ccc(Cl)cc1]

Following general procedure A using methyl 4-chlorobenzoate (170.6 mg, 1.0 mmol), the reaction was carried out for 18 h. The reaction solution was cooled to 0° C. and diluted with MeOH (2.5 mL). NaBH₄ (56.7 mg, 1.5 equiv) was added and the reaction solution stirred at 0° C. for 15 min. The mixture was warmed to room temperature, stirred for 15 min, then heated to 65° C. for 4 h [Caution: vigorous gas evolution was observed!]. The solution was cooled to room temperature and quenched with ca. 5 mL of saturated aqueous NaHCO₃. The aqueous layer was washed three times with ca. 5 mL Et₂O. The combined organic layers were dried over MgSO₄, vacuum filtered, and concentrated in vacuo with the aid of a rotary evaporator. The crude residue was then purified with the aid of CombiFlash Nextgen 300 (12 g gold cartridge, elutes using 5% EtOAc/1% NEt₃ in Hexanes at 30 mL/min). The title compound was obtained as a yellow oil (118.7 mg, 60% yield).

The spectroscopic data for this compound match those previously reported in the literature.

¹H NMR (600 MHZ, CDCl₃) δ 7.30-7.23 (m, 4H), 3.75 (s, 2H), 2.63-2.58 (m, 2H), 1.49 (dt, J=14.4, 7.4 Hz, 2H), 1.40 (br s, 1H), 1.35 (dq, J=14.4, 7.3 Hz, 2H), 0.91 (t, J=7.4 Hz, 3H).

¹³C NMR (151 MHZ, CDCl₃) δ 139.2, 132.6, 129.5, 128.6, 53.5, 49.2, 32.3, 20.6, 14.1.

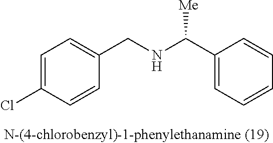 [SMILES: C[C@@H](NCc1ccc(Cl)cc1)c1ccccc1]

Following general procedure A using methyl 4-chlorobenzoate (170.6 mg, 1.0 mmol) and (R)-1-phenylethanamine (216.4 μL, 1.7 mmol, 1.7 equiv), the reaction was carried out for 21 h. The reaction solution was cooled to 0° C. and diluted with MeOH (2.5 mL). NaBH₄ (56.7 mg, 1.5 equiv) was added, and the reaction solution stirred at 0° C. for 15 min. The mixture was warmed to room temperature, stirred for 15 min, then heated to 65° C. for 9 h [Caution: vigorous gas evolution was observed!]. The solution was cooled to room temperature and quenched with ca. 5 mL of saturated aqueous NaHCO₃. The aqueous layer was washed three times with ca. 5 mL Et₂O. The combined organic layers were dried over MgSO₄, vacuum filtered, and concentrated in vacuo with the aid of a rotary evaporator. The crude residue was then purified with the aid of CombiFlash Nextgen 300 (12 g gold cartridge, elutes using 10% EtOAc/1% NEt₃ in Hexanes at 30 mL/min). The title compound was obtained as a pale yellow oil (141.8 mg, 58% yield).

The spectroscopic data for this compound match those previously reported in the literature.

¹H NMR (600 MHZ, CDCl₃) δ 7.29-7.24 (m, 4H), 7.21-7.16 (m, 3H), 7.14 (app d, J=8.3 Hz, 2H), 3.70 (q, J=6.6 Hz, 1H), 3.57-3.44 (m, 2H), 1.50 (br s, 1H), 1.29 (d, J=6.6, 3H).

¹³C NMR (151 MHZ, CDCl₃) δ 145.5, 139.3, 132.6, 129.6, 128.6, 128.6. 127.1, 126.8, 57.6, 51.0, 24.6.

HPLC: Chiralcel OJ-H column (Hexane/i-PrOH 98/2, flow rate 0.7 mL/min, 5.00 μL injection, λ=210 nm), t_{R}=21.77 min.

Synthesis of rac-19: A flame-dried 50 mL 2-neck round bottom flask was equipped with a magnetic stir. The flask was charged with 4 Å molecular sieves (2.0 g) and 4-chlorobenzaldehyde (0.70 g, 5.0 mmol, 1.0 equiv) The flask and reflux apparatus were evacuated and backfilled with nitrogen (this process was repeated to a total of three times). PhMe (5.0 mL, 1.0 M) and rac-1-phenylethanamine (0.97 mL, 7.5 mL, 1.5 equiv) were injected and the reaction solution was heated to 65° C. for 19 h. Once the reaction was complete, the reaction solution was filtered over a pad of celite and concentrated in vacuo with the aid of a rotary evaporator. The crude residue was added to a flame-dried 50 mL 2-neck round bottom flask equipped with a magnetic stir bar. MeOH (10.0 mL, 0.05 M) was injected into the flask and the solution was cooled to 0° C. NaBH₄ (226.8 mg, 6.0 mmol, 1.2 equiv) was added to the flask. The reaction solution stirred at 0° C. for 15 min. The mixture was warmed to room temperature, stirred for 15 min, then heated to 50° C. for 9 h [Caution: vigorous gas evolution was observed]. The solution was cooled to room temperature and quenched with ca. 10 mL of saturated aqueous NaHCO₃. The aqueous layer was washed three times with ca. 10 mL Et₂O. The combined organic layers were dried over MgSO₄, vacuum filtered, and concentrated in vacuo with the aid of a rotary evaporator. The crude residue was then purified with the aid of CombiFlash Nextgen 300 (12 g gold cartridge, elutes using 10% EtOAc/1% NEt₃ in Hexanes at 30 mL/min). The title compound was obtained as a colorless oil.

HPLC: Chiralcel OJ-H column (Hexane/i-PrOH 98/2, flow rate 0.7 mL/min, 5.00 μL injection, λ=210 nm), t_{R} (left)=19.18, t_{R} (right)=21.70 min.

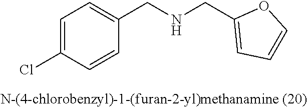 [SMILES: Clc1ccc(CNCc2ccco2)cc1]

Following general procedure A using methyl 4-chlorobenzoate (170.6 mg, 1.0 mmol) and furfurylamine (157.2 μL, 1.7 mmol, 1.7 equiv) in lieu of n-butylamine, the reaction was carried out for 21 h. The reaction solution was cooled to 0° C. and diluted with MeOH (2.5 mL). NaBH₄ (56.7 mg, 1.5 equiv) was added and the reaction solution stirred at 0° C. for 15 min. The mixture was warmed to room temperature, stirred for 15 min, then heated to 65° C. for 4 h [Caution: vigorous gas evolution was observed!]. The solution was cooled to room temperature and quenched with ca. 5 mL of saturated aqueous NaHCO₃. The aqueous layer was washed three times with ca. 5 mL Et₂O. The combined organic layers were dried over MgSO₄, vacuum filtered, and concentrated in vacuo with the aid of a rotary evaporator. The crude residue was then purified with the aid of CombiFlash Nextgen 300 (12 g gold cartridge, elutes using 5→10% EtOAc/1% NEt₃ in Hexanes at 30 mL/min). The title compound was obtained as a yellow oil (142.7 mg, 64% yield).

The spectroscopic data for this compound match those previously reported in the literature.

¹H NMR (600 MHZ, CDCl₃) δ 7.35 (dd, J=1.8, 0.9 Hz, 1H), 7.29-7.22 (m, 4H), 6.30 (dd, J=3.2, 1.8 Hz, 1H), 6.15 (dd, J=3.1, 0.8 Hz, 1H), 3.74 (s, 2H), 3.73 (s, 2H), 1.71 (br s, 1H).

¹³C NMR (151 MHZ, CDCl₃) δ 153.8, 142.0, 138.5, 132.8, 129.7, 128.6, 110.3, 107.3, 52.1, 45.4.

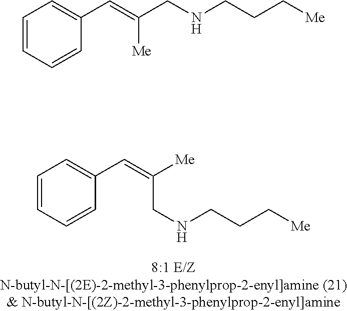 [SMILES: CCCCNC/C(C)=C/c1ccccc1 ; CCCCNC/C(C)=C\c1ccccc1]

Following general procedure A using methyl 3-methyl-3-phenylacrylate (88.1 mg, 0.5 mmol), the reaction was carried out for 20 h. The reaction solution was cooled to 0° C. and diluted with MeOH (2.5 mL). NaBH₄ (56.7 mg, 3.0 equiv) was added and the reaction solution stirred at 0° C. for 15 min. The mixture was warmed to room temperature, stirred for 15 min, then heated to 65° C. for 4 h [Caution: vigorous gas evolution was observed]. The solution was cooled to room temperature and quenched with ca. 5 mL of saturated aqueous NaHCO₃. The aqueous layer was washed three times with ca. 5 mL Et₂O. The combined organic layers were dried over MgSO₄, vacuum filtered, and concentrated in vacuo with the aid of a rotary evaporator. The crude residue was then purified with the aid of CombiFlash Nextgen 300 (12 g gold cartridge, elutes using 5% EtOAc/1% NEt₃ in Hexanes at 30 mL/min). The title compound was obtained as a yellow oil (72.9 mg, 72% yield).

The spectroscopic data for (E)-21 match those previously reported in the literature.

(Z)-21 was assigned by analogy to N-propyl-N-[(2E)-2-methyl-3-phenylprop-2-enyl] amine.

¹H NMR (500 MHZ, CDCl₃) δ 7.35-7.30 (m, 2H), 7.27 (d, J=7.9 Hz, 2H), 7.22-7.18 (m, 1H), 6.44 (s, 0.8H), 6.42 (s, 0.2H), 3.38 (s, 0.2H), 3.33 (s, 1.8H), 2.64 (t, J=7.3, 1.8H), 2.54 (t, J=7.2 Hz, 0.2H), 1.95 (d, J=1.6 Hz, 0.3H), 1.90 (d, J=1.5 Hz, 2.6H), 1.56-1.48 (m, 2H), 1.45-1.29 (m, 3H), 0.93 (t, J=7.3 Hz, 2.6H), 0.89 (t, J=7.3 Hz, 0.4H).

¹³C NMR trans (major) (151 MHz, CDCl₃) δ 138.2, 137.3, 129.0, 128.2, 126.2, 125.6, 58.3, 49.1, 32.4, 20.7, 16.7, 14.2.

¹³C NMR cis (minor) (151 MHZ, CDCl₃) δ 138.0, 137.6, 128.8, 128.2, 127.9, 126.3, 50.6, 49.3, 32.3, 23.0, 20.6, 14.1.

HRMS (+ESI): Calculated for C₁₄H₂₁N [M+H]: 204.1747. Found: 204.1724.

LRMS (GC-MS): Calculated for C₁₄H₂₁N: 203.17. Found: 203.15 (peak 1) and 203.15 (peak 2).

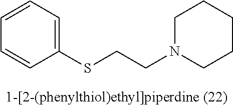 [SMILES: c1ccc(SCCN2CCCCC2)cc1]

Following general procedure B using Methyl 2-(phenylthiol)acetate (182.2 mg, 1.0 mmol), the reaction was carried out for 24 h. The reaction solution was cooled to 0° C. and diluted with MeOH (2.5 mL). NaBH₄ (113.5 mg, 3.0 equiv) was added and the reaction solution stirred at 0° C. for 15 min. The mixture was warmed to room temperature, stirred for 15 min, then heated to 65° C. for 9 h. The solution was cooled to room temperature and quenched with ca. 5 mL of saturated aqueous NaHCO₃. The aqueous layer was washed three times with ca. 5 mL Et₂O. The combined organic layers were dried over MgSO₄, vacuum filtered, and concentrated in vacuo with the aid of a rotary evaporator. The crude residue was then purified with the aid of CombiFlash Nextgen 300 (12 g gold cartridge, elutes using 5% EtOAc/1% NEt₃ in Hexanes at 30 mL/min). The title compound was obtained as a yellow oil (144.3 mg, 65% yield).

The spectroscopic data for this compound are in good agreement with those previously reported in the literature.

¹H NMR (600 MHZ, CDCl₃) δ 7.29-7.25 (m, 2H), 7.23-7.17 (m, 2H), 7.09 (t, J=7.4 Hz, 1H), 3.04-2.95 (m, 2H), 2.58-2.49 (m, 2H), 2.35 (br s, 4H), 1.51 (p, J=5.6 Hz, 4H), 1.34-1.38 (m, 2H).

¹³C NMR (151 MHZ, CDCl₃) δ 136.7, 129.0, 129.0, 125.9, 58.6, 54.6, 30.8, 26.0, 24.4.

IR (neat): 2932, 2757, 1584, 1480, 1439, 1104, 735, 689, 474 cm⁻¹.

HRMS (+ESI): Calculated for C₁₃H₁₉NS [M+H]: 222.1311. Found: 222.1295.

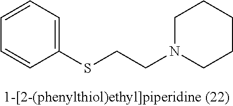 [SMILES: c1ccc(SCCN2CCCCC2)cc1]

Amine 22 was obtained following general procedure A with the following modifications: using methyl 3-cyclohexylpropionate (170.3 mg, 1.0 mmol), N-benzylamine (327.7 μL, 3.0 equiv), and DMMS (0.74 mL, 6.0 equiv), the reaction was carried out for 22 h. The reaction solution was cooled to 0° C. and diluted with MeOH (2.5 mL). NaBH₄ (113.5 mg, 3.0 equiv) was added and the reaction solution stirred at 0° C. for 15 min. The mixture was warmed to room temperature, stirred for 15 min, then heated to 65° C. for 4 h. The solution was cooled to room temperature and quenched with ca. 5 mL of saturated aqueous NaHCO₃. The aqueous layer was washed three times with ca. 5 mL Et₂O. The combined organic layers were dried over MgSO₄, vacuum filtered, and concentrated in vacuo with the aid of a rotary evaporator. The crude residue was then purified with the aid of CombiFlash Nextgen 300 (12 g gold cartridge, elutes using 5% EtOAc/1% NEt₃ in Hexanes at 30 mL/min). The title compound was obtained as a viscous, pale yellow oil (169.9 mg, contaminated with ca. 3% N,N-dibenzylamine, 71% yield).

The spectroscopic data for this compound match those previously reported in the literature.

¹H NMR (600 MHZ, CDCl₃) δ 7.35-7.31 (m, 4H), 7.27-7.24 (m, 1H), 3.80 (s, 2H), 2.61 (t, J=7.3 Hz, 2H), 1.87 (br s, 1H), 1.74-1.65 (m, 4H), 1.65-1.61 (m, 1H), 1.55-1.50 (m, 2H), 1.28-1.17 (m, 5H), 1.17-1.09 (m, 1H), 0.92-0.82 (m, 2H).

¹³C NMR (151 MHZ, CDCl₃) δ 140.3, 128.5, 128.3, 127.1, 54.1, 49.9, 37.7, 35.2, 33.5, 27.4, 26.8, 26.5.

### Example 3

Additional experiments were performed relating to reaction optimization and substrates.

General Procedure for Reaction Optimization (Using an Aryl Ester): Cp₂ZrCl₂ (2.9 mg, 5.0 mol %) and methyl 4-chlorobenzoate (34.1 mg, 0.2 mmol) were weighed into a flame-dried 2-dram vial equipped with a magnetic stir bar. The vial was capped with an open top screw cap equipped with a Teflon-lined silicon septum and sealed with electrical tape. The vial was then evacuated and backfilled with nitrogen (this process was repeated to a total of three times). The solids were dissolved in 0.5 mL of anhydrous PhMe (0.4 M). DEMS (95.9 μL, 3.0 equiv) was injected into the reaction mixture, followed by the injection of n-butylamine (33.7 μL, 1.7 equiv) using a Hamilton gastight glass microsyringe. The septum of the reaction tube was sealed with wax and the reaction solution was stirred at 700 rpm at 80° C. for 16-24 h. The reaction solution was quenched with ca. 1 mL of 1 M HCl and left to stir at 700 rpm at 80° C. for approx. 1 h. The solution was diluted with ca. 2 mL of CH₂Cl₂ and ca. 2 mL H₂O. The aqueous layer was washed three times with ca. 3 mL CH₂Cl₂. The combined organic layers were dried over Na₂SO₄, vacuum filtered, and concentrated in vacuo with the aid of a rotary evaporator. A ¹H NMR yield was obtained using mesitylene (13.9 μL, 0.1 mmol) as an internal standard.

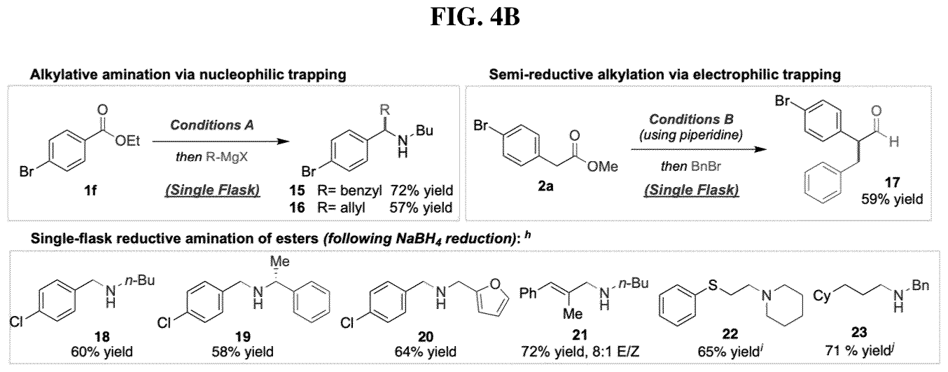

FIG. 5 shows a scheme and results for screening of amines for the semi-reduction of methyl 4-chlorobenzoate.

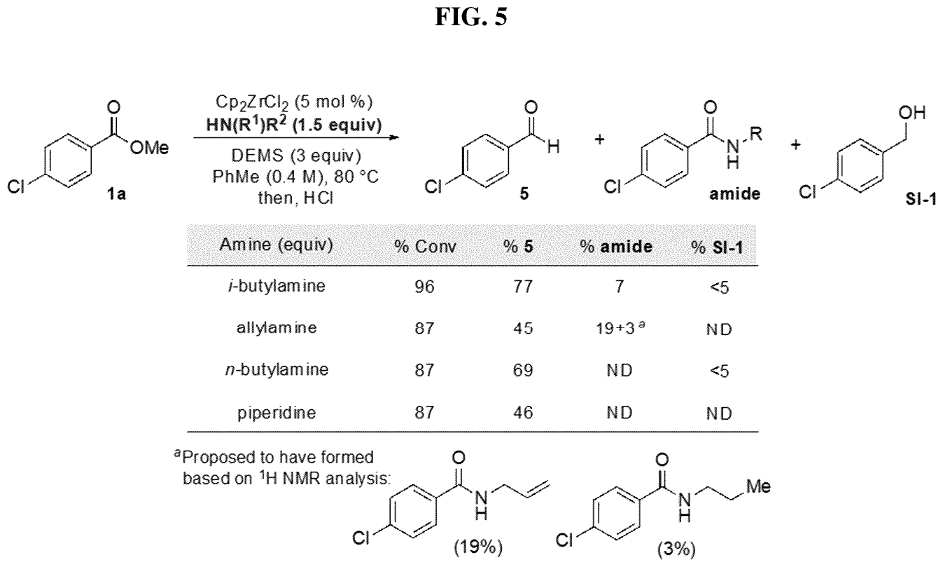

FIG. 6 shows a scheme and results of control experiments for the semi-reduction of methyl 4-chlorobenzonate.

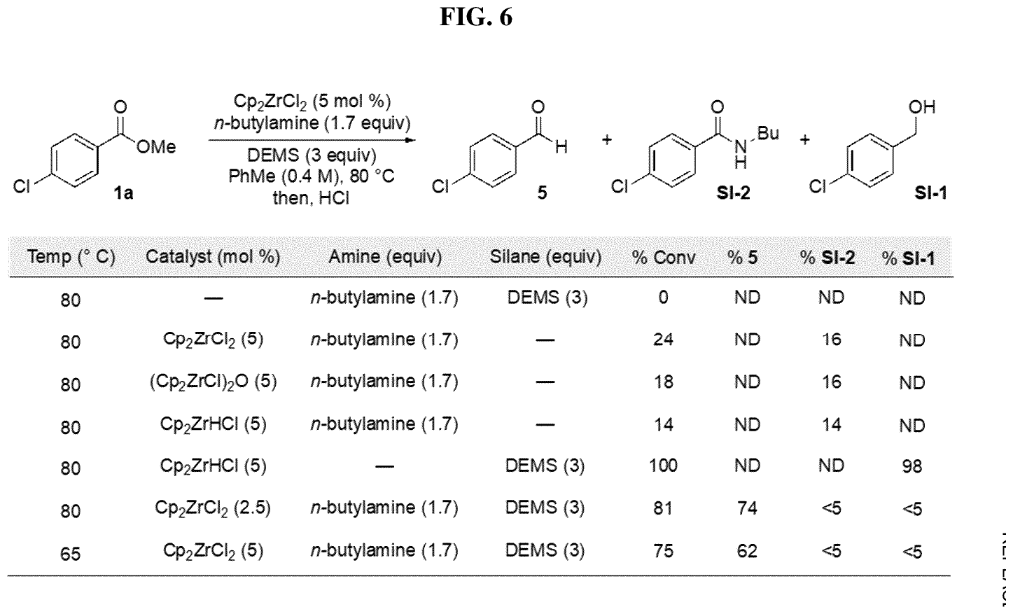

FIG. 7 shows a scheme and results for testing amidation in the presence of 20 mol % DEMS.

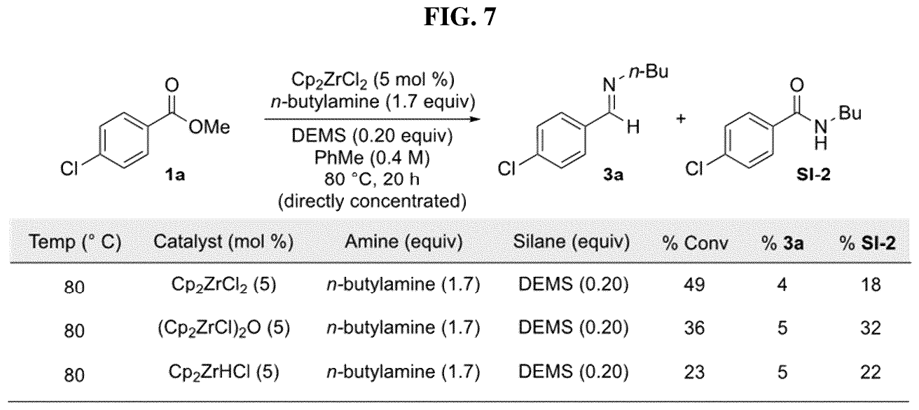

FIG. 8 shows a scheme and results for screening of metallocenes for the semi-reduction of methyl 4-chlorobenzoate.

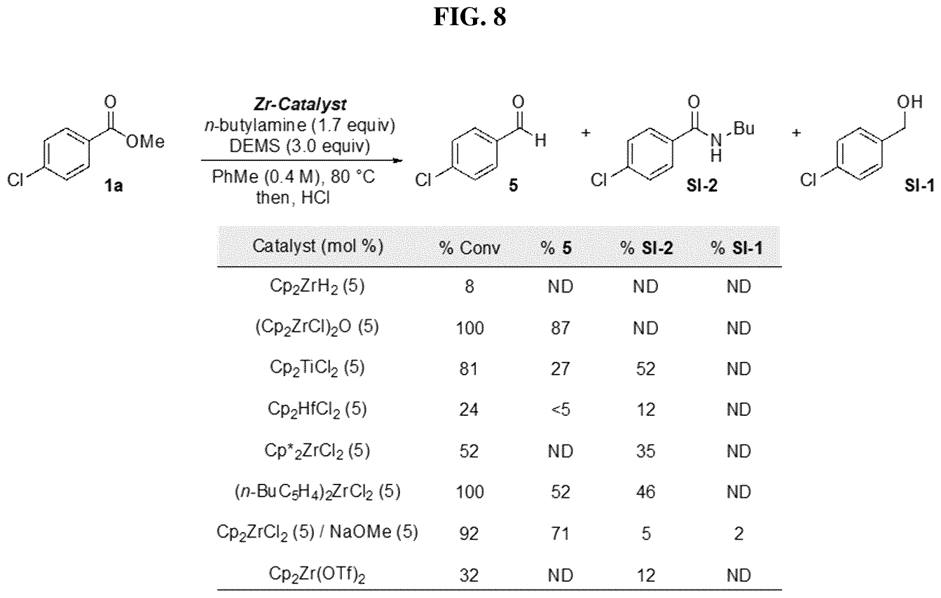

FIG. 9 shows a scheme and results for extended optimization of the information shown in FIG. 3.

General Procedure for Reaction Optimization (Using an Aliphatic Ester): Cp₂ZrCl₂ (5.8 mg, 10.0 mol %) was weighed into a flame-dried 2-dram vial equipped with a magnetic stir bar. The vial was capped with an open top screw cap equipped with a Teflon-lined silicon septum and sealed with electrical tape. The vial was then evacuated and backfilled with nitrogen (this process was repeated to a total of three times). The catalyst was dissolved in 1.0 mL of anhydrous PhMe (0.2 M), followed by the injection of piperidine (33.3 μL, 0.34 mmol, 1.7 equiv) using a Hamilton gastight glass microsyringe. Methyl 4-bromophenylacetate (45.8 mg, 0.2 mmol, 1.0 equiv) and DEMS (95.9 μL, 3.0 equiv) were injected into the reaction mixture via microsyringe. The septum of the reaction vial was sealed with wax and the reaction solution was stirred at 700 rpm at 80° C. for 17-21 h. The reaction solution was quenched with ca. 1 mL of 1 M HCl and left to stir at 700 rpm at 80° C. for approx. 1 h. The solution was diluted with ca. 2 mL of CH₂Cl₂ and ca. 2 mL H₂O. The aqueous layer was washed three times with ca. 3 mL CH₂Cl₂. The combined organic layers were dried over Na₂SO₄, vacuum filtered, and concentrated in vacuo with the aid of a rotary evaporator. A ¹H NMR yield was obtained using mesitylene (13.9 μL, 0.1 mmol) as an internal standard.

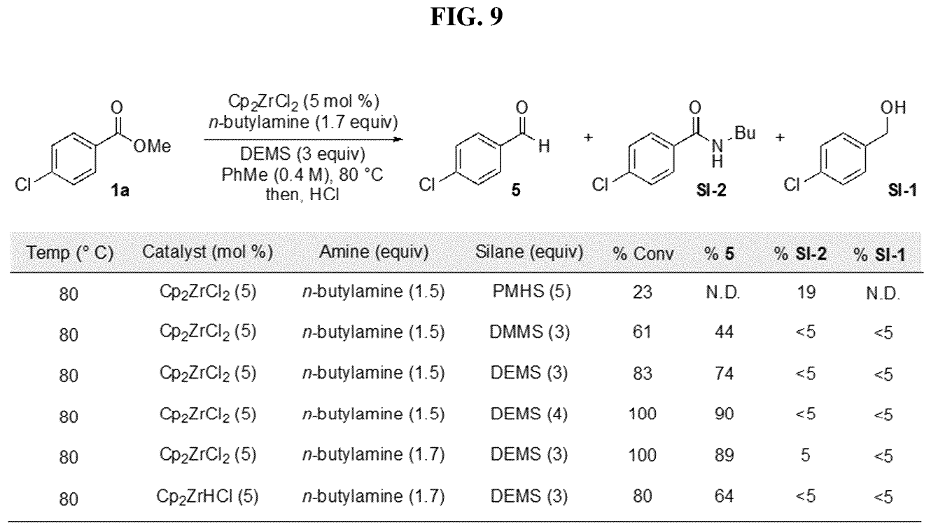

FIG. 10 shows a scheme and results of extended optimization for the semi-reduction of methyl 2-(4-bromophenyl)acetate.

### Example 4

FIG. 11A shows a scheme for synthesis of aldehyde 24 using a clear plastic bottle. In FIGS. 11A, 11B, and 11C, reported values are based on the theoretical amount of repeating monomer unit. The polyester (PET) starting material from a water bottle was cut into small pieces and ground with dry ice using an electrical grinder. The ground PET was then rinsed with acetone and dried under high vacuum at room temperature overnight. A 25 mL Schlenk flask with a magnetic stirring bar was dried in an oven (140° C.) overnight and then transferred into a glovebox with a stopper. PET (192.2 mg, ca. 1.0 mmol, 1.0 equiv), Cp₂ZrC₁₂ (29.2 mg, 0.1 mmol, 0.1 equiv, 15.2 weight %) and PhMe (10 mL, 0.1 M) were added to the flask. DMMS (1.46 mL, 12.0 mmol, 12.0 equiv, 6.6 weight equiv) and n-butylamine (791 μL, 8.0 mmol, 8.0 equiv, 3.0 weight equiv) were slowly added to the mixture with stirring [gas evolution was observed]. The solution was left to stir for approx. 5 minutes until no obvious bubbles were observed. The flask was then sealed with a stopper and removed from the glovebox. It was connected to N₂ flow and stirred at 80° C. for 20 hours. After cooling to room temperature, the reaction solution was transferred to a 100 mL round bottom flask and rinsed with CH₂Cl₂. The solvents were evaporated in vacuo. The mixture was diluted with approx. 10 mL 4 M HCl and approx. 20 mL H₂O and stirred at room temperature. The mixture was diluted with brine and extracted with DCM (3×10 mL). The organic layer was collected and dried with anhydrous MgSO₄, followed by concentration in vacuo using a rotary evaporator. The crude residue was purified via silica gel chromatography (elutes using a 20% EtOAc/Hexane eluent). The dialdehyde was isolated as a white powder (111.3 mg, 83% yield).

The spectroscopic data for this compound match those previously reported in the literature.

¹H NMR (600 MHZ, CDCl₃) δ 10.14 (s, 2H), 8.06 (s, 4H).

¹³C NMR (151 MHz, CDCl₃) δ 191.6, 140.2, 130.3.

Synthesis of aldehyde 24 using a green beverage bottle: The polyester (PET) starting material from a green plastic bottle was cut into small pieces and ground with dry ice using an electrical grinder. The ground PET was then rinsed with acetone and dried under high vacuum at room temperature overnight. A 25 mL Schlenk flask with a magnetic stirring bar was dried in an oven (140° C.) overnight and then transferred into a glovebox with a stopper. PET (192.2 mg, ca. 1.0 mmol, 1.0 equiv), Cp₂ZrCl₂ (29.2 mg, 0.1 mmol, 0.1 equiv, 15.2 weight %) and PhMe (10 mL, 0.1 M) were added to the flask. DMMS (1.46 mL, 12.0 mmol, 12.0 equiv, 6.6 weight equiv) and n-butylamine (791 μL, 8.0 mmol, 8.0 equiv, 3.0 weight equiv) were slowly added to the mixture with stirring [gas evolution was observed]. The solution was left to stir for approx. 5 minutes until no obvious bubbles were observed. The flask was then sealed with a stopper and removed from the glovebox. It was connected to N₂ flow and stirred at 80° C. for 20 hours. After cooling to room temperature, the reaction solution was transferred to a 100 mL round bottom flask and rinsed with CH₂Cl₂. The solvents were evaporated in vacuo. The mixture was diluted with approx. 10 mL 4 M HCl and approx. 20 mL H₂O and stirred at room temperature. The mixture was diluted with brine and extracted with DCM (3×10 mL). The organic layer was collected and dried with anhydrous MgSO₄, followed by concentration in vacuo using a rotary evaporator. The crude residue was purified via silica gel chromatography (elutes using a 20% EtOAc/Hexane eluent). The dialdehyde was rinsed with hexane and isolated as a white powder (85.4 mg, 64% yield).

Synthesis of aldehyde 24 using a polyester T-shirt: The polyester (PET) fabric starting material from a T-shirt was cut into small pieces and ground with dry ice using an electrical grinder. The ground PET was then rinsed with acetone and dried under high vacuum at room temperature overnight. A 100 mL Schlenk flask with a magnetic stirring bar was dried in an oven (140° C.) overnight and then transferred into a glovebox with a stopper. PET (192.2 mg, ca. 1.0 mmol, 1.0 equiv), Cp₂ZrCl₂ (29.2 mg, 0.1 mmol, 0.1 equiv, 15.2 weight %) and PhMe (10 mL, 0.1 M) were added to the flask. DMMS (1.46 mL, 12.0 mmol, 12.0 equiv, 6.6 weight equiv) and n-butylamine (791 μL, 8.0 mmol, 8.0 equiv, 3.0 weight equiv) were slowly added to the mixture with stirring [gas evolution was observed]. The solution was left to stir for 5 minutes until no obvious bubbles were observed. The flask was then sealed with a stopper and removed from the glovebox. It was connected to N₂ flow and stirred at 80° C. for 20 hours. After cooling to room temperature, the reaction solution was quenched with approx. 10 mL 4 M HCl and approx. 20 mL H₂O and stirred at room temperature. The mixture was diluted with brine and extracted with DCM (3×10 mL). The organic layer was collected and dried with anhydrous MgSO₄, followed by concentration in vacuo using a rotary evaporator. The crude residue was purified via silica gel chromatography (elutes using a 20% EtOAc/Hexane eluent). The dialdehyde was isolated as a white powder (122.0 mg, 91% yield).

Synthesis of aldehyde 24 using a laptop screen protector: The polyester (PET) fabric starting material from screen protector was cut into small pieces and ground with dry ice using an electrical grinder. The ground PET was then rinsed with acetone and dried under high vacuum at room temperature overnight. A 100 mL Schlenk flask with a magnetic stirring bar was dried in an oven (140° C.) overnight and then transferred into a glovebox with a stopper. PET (192.2 mg, ca. 1.0 mmol, 1.0 equiv), Cp₂ZrCl₂ (29.2 mg, 0.1 mmol, 0.1 equiv, 15.2 weight %) and PhMe (10 mL, 0.1 M) were added to the flask. DMMS (1.46 mL, 12.0 mmol, 12.0 equiv, 6.6 weight equiv) and n-butylamine (791 μL, 8.0 mmol, 8.0 equiv, 3.0 weight equiv) were slowly added to the mixture with stirring [gas evolution was observed]. The solution was left to stir for 5 minutes until no obvious bubbles were observed. The flask was then sealed with a stopper and removed from the glovebox. It was connected to N₂ flow and stirred at 80° C. for 20 hours. After cooling to room temperature, the reaction solution was transferred to a 100 mL round bottom flask and rinsed with CH₂Cl₂. The solvents were evaporated in vacuo. The mixture was diluted with approx. 10 mL 4 M HCl and approx. 20 mL H₂O and stirred at room temperature. The mixture was diluted with brine and extracted with DCM (3×10 mL). The organic layer was collected and dried with anhydrous MgSO₄, followed by concentration in vacuo using a rotary evaporator. The crude residue was purified via silica gel chromatography (elutes using a 20% EtOAc/Hexane eluent). The dialdehyde was rinsed with hexane and isolated as a white powder (65.4 mg, 48% yield).

FIG. 11B shows a scheme for depolymerization of polyethylene terephthalate without hydrolysis.

Synthesis of diimine 25 using a clear plastic bottle: The polyester (PET) starting material from a water bottle was cut into small pieces and ground with dry ice using an electrical grinder. The ground PET was then rinsed with acetone and dried under high vacuum at room temperature overnight. A 25 mL Schlenk flask with a magnetic stirring bar was dried in an oven (140° C.) overnight and then transferred into a glovebox with a stopper. PET (192.2 mg, ca. 1.0 mmol, 1.0 equiv), Cp₂ZrCl₂ (29.2 mg, 0.1 mmol, 0.1 equiv, 15.2 weight %) and PhMe (10 mL, 0.1 M) were added to the flask. DMMS (1.46 mL, 12.0 mmol, 12.0 equiv, 6.6 weight equiv) and n-butylamine (791 μL, 8.0 mmol, 8.0 equiv, 3.0 weight equiv) were slowly added to the mixture with stirring [gas evolution was observed]. The solution was left to stir for approx. 5 minutes until no obvious bubbles were observed. The flask was then sealed with a stopper and removed from the glovebox. It was connected to N₂ flow and stirred at 80° C. for 20 hours. After cooling to room temperature, the reaction solution was directly purified via flash column chromatography using activated aluminum oxide (neutral, Brockmann Grade I, 58 Å) (elutes using 5% EtOAc/1% NEt₃ in Hex). The title compound was obtained as a colorless oil (134 mg, 55% yield).

The spectroscopic data for this compound match those previously reported in the literature.

¹H NMR (600 MHZ, CDCl₃) δ 8.28 (s, 2H), 7.75 (s, 4H), 3.62 (td, J=7.0, 1.3 Hz, 4H), 1.73-1.65 (m, 4H), 1.39 (sex, J=7.4 Hz, 4H), 0.98-0.91 (m, 6H).

¹³C NMR (151 MHZ, CDCl₃) δ 160.3, 138.2, 128.3, 61.7, 33.1, 20.6, 14.0.

FIG. 11C shows a scheme for the depolymerization of poly(ethylene succinate).

Synthesis of pyrrole 26: A 25 mL Schlenk flask with a magnetic stirring bar was dried in an oven (140° C.) overnight and then transferred into a glovebox with a stopper. Poly(ethylene succinate) (144 mg, ca. 1.0 mmol, 1.0 equiv), Cp₂ZrCl₂ (29.2 mg, 0.1 mmol, 0.1 equiv, 20.3 weight %) and PhMe (10 mL, 0.1 M) were added to the flask. DMMS (1.46 mL, 12.0 mmol, 12.0 equiv, 8.8 weight equiv) and N-benzylamine (874 μL, 8.0 mmol, 8.0 equiv, 6.0 weight equiv) were slowly added to the mixture with stirring [gas evolution was observed]. The solution was left to stir for 5 to 10 minutes until no obvious bubbles were observed. The flask was then sealed with a stopper and removed from the glovebox. It was connected to N₂ flow and stirred at 80° C. for 20 hours. The reaction solution was cooled to room temperature and directly concentrated in vacuo. The title product was obtained by silica gel chromatography (flushed with ca. 100 mL 100% Hex, then elutes using a 5% EtOAc/Hexane eluent). The N-benzylpyrrole was isolated as a colorless oil (134.4 mg, 85% yield).

The spectroscopic data for this compound match those previously reported in the literature.

¹H NMR (600 MHZ, CDCl₃) δ 7.36-7.33 (m, 2H), 7.32-7.27 (m, 1H), 7.14 (d, J=7.4 Hz, 2H), 6.72-6.71 (m, 2H), 6.24-6.20 (m, 2H), 5.09 (s, 2H).

¹³C NMR (151 MHZ, CDCl₃) δ 138.3, 128.8, 127.8, 127.1, 121.3, 108.6, 53.5.

### Example 5

The profound effect on product chemoselectivity imparted by a simple unprotected amine prompted the investigation of the mechanism of this interrupted ZrH-catalyzed ester reduction. Prior reports regarding the interconversion of esters to amides mediated by Lewis acidic zirconocenes suggested that a similar mechanistic pathway might be involved, the products of which would be amenable to ZrH-catalyzed partial reduction. Of note, varying quantities of amide byproducts were observed throughout the course of the studies. However, experimental mechanistic investigations suggest that this is unlikely the sole or major route of conversion.

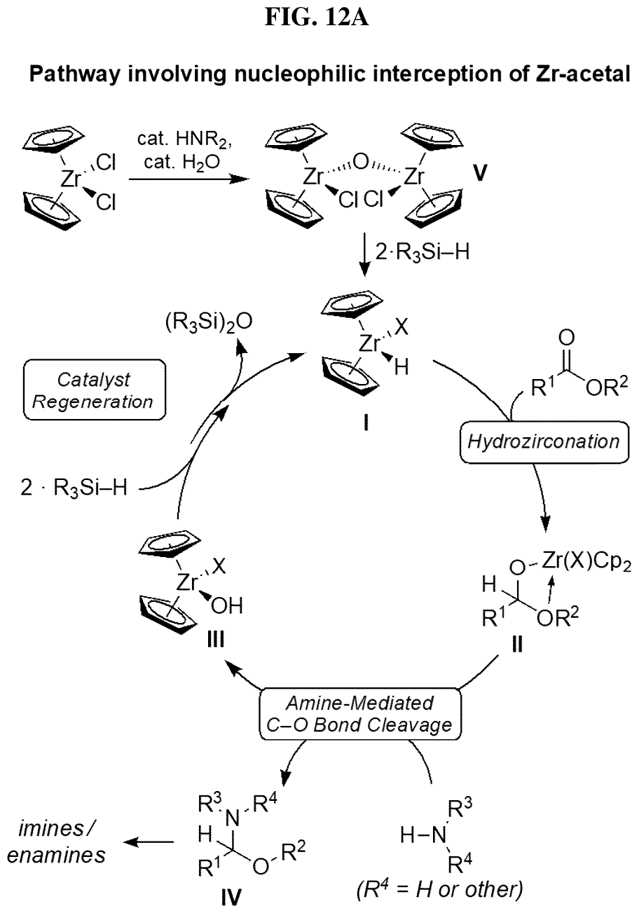

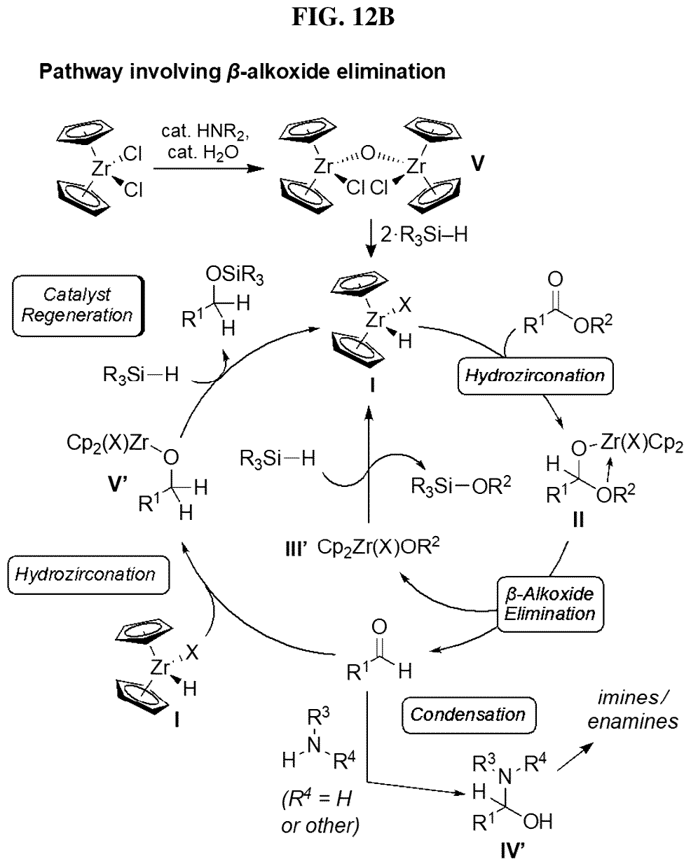

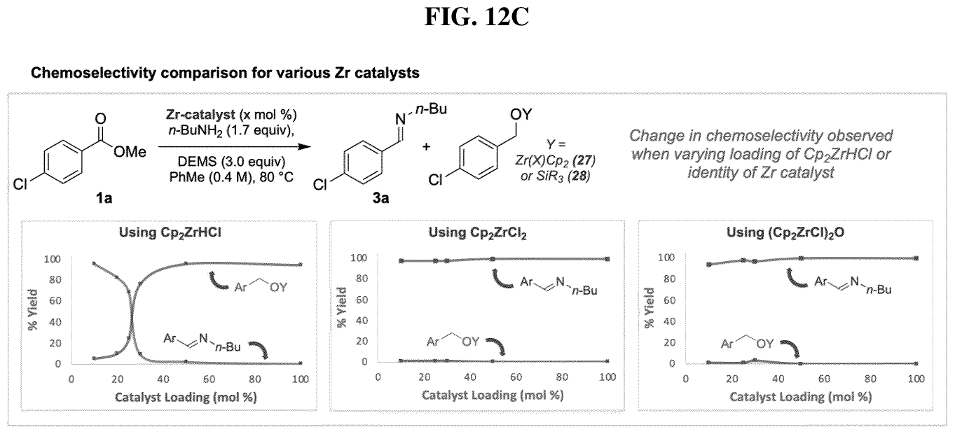

FIGS. 12A and 12B show plausible mechanistic pathways. It was considered that the dominant mechanistic pathway leading to product formation could involve zirconocene hemiacetal II (FIGS. 12A and 12B). This species may be directly intercepted by an exogeneous amine, analogous to prior observations with related hemiaminals (FIG. 12A). Alternatively, upon B-alkoxy elimination, interception of the resulting aldehyde by the amine may simply outpace continued reduction to the alcohol (FIG. 12B) (e.g., I to V′). In either scenario, the resulting putative hydroxyzirconocene or alkoxyzirconocene species, III/III′, could regenerate active catalyst I through hydrosilane-meditate metathesis.

To gain further mechanistic insight, a series of experiments was performed with varying ZrH sources and loadings. Initial attempts to hydrozirconate 1a using 1.0 equivalent of Cp₂ZrHCl in the presence of n-butylamine did not result in the formation of imine 3a, even in trace quantities. Rather, within 30 minutes 1a was iteratively reduced to produce a zirconocene alkoxide 27. While studying the ester reduction at various catalyst loadings, distinct differences in reaction outcome were discovered (FIG. 12C). Reactions employing ≤25 mol % Cp₂ZrHCl exhibited pronounced selectivity for imine 3a, while those employing ≥30 mol % Cp₂ZrHCl sharply favored full reduction to 27 and 28. Conversely, this effect was not observed when carrying out an analogous study employing either Cp₂ZrCl₂ or (Cp₂ZrC₁) 20 (V) pre-catalysts instead. In both studies, the major product observed at all catalyst loadings was imine 3a. These findings suggest that the active catalyst for semi-reduction may involve a ZrH complex I where X≠Cl. This “X” ligand could be an alkoxide or siloxide, or that the active catalyst could be dimeric in nature.

### Example 6

FIG. 13 shows schemes for general procedures A, B, and C for preparation of starting materials.

Starting Material General Procedure A: A flame-dried 50 mL 2-neck round bottom flask was equipped with a magnetic stir bar and reflux apparatus. The carboxylic acid (10.0 mmol) was added to the flask. The flask and reflux apparatus were evacuated and backfilled with nitrogen (this process was repeated to a total of three times). The alcohol (20 mL, 0.5 M) and H₂SO₄ (0.4 mL) were injected and the reaction solution was heated to reflux for 18-24 h. Once the reaction was complete, the reaction solution was quenched with H₂O (approx. 10 mL) and diluted with CH₂Cl₂ (approx. 10 mL). The aqueous layer was washed three times with CH₂Cl₂, dried over Na₂SO₄, vacuum filtered, and concentrated in vacuo with the aid of a rotary evaporator. The crude residue was purified by automated column chromatography to afford the desired ester.

Starting Material General Procedure B: A flame-dried 50 mL 2-neck round bottom flask equipped with a magnetic stir bar was charged with carboxylic acid (10.0 mmol). The flask was evacuated and backfilled with nitrogen (this process was repeated to a total of three times). DMF (0.5 M), was injected into the flask. K₂CO₃ (2.0 equiv) was added to the flask, followed by the dropwise addition of Mel (2.0 equiv). The reaction was left to stir at room temperature. Once the reaction was complete, the reaction solution was quenched with H₂O (approx. 10 mL) and diluted with EtOAc (approx. 10 mL). The aqueous layer was washed with EtOAc, dried over Na₂SO₄, vacuum filtered, and concentrated in vacuo with the aid of a rotary evaporator. The crude residue was purified by automated column chromatography to afford the desired ester.

Starting Material General Procedure C: A flame-dried 50 mL round bottom flask was equipped with a magnetic stir bar. CH₂Cl₂ (0.4 M) was injected into the flask, followed by the injection of the alcohol (3.0 equiv). NEt₃ (3.0 equiv) was added to the flask, followed by the dropwise or portionwise addition of acid chloride (1.0 equiv). The reaction was left to stir at room temperature. Once the reaction was complete, the reaction solution was quenched with H₂O (approx. 10 mL) and diluted with CH₂Cl₂ (approx. 10 mL). The aqueous layer was washed with CH₂Cl₂, dried over Na₂SO₄, vacuum filtered, and concentrated in vacuo with the aid of a rotary evaporator. The crude residue was purified by automated column chromatography to afford the desired ester.

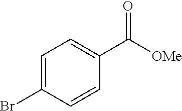 [SMILES: COC(=O)c1ccc(Br)cc1]

(1d) Methyl 4-bromobenzoate was obtained following the Starting Material General Procedure C. The spectroscopic data for this compound match those previously reported in the literature.

¹H NMR (400 MHZ, CDCl₃) δ 7.93-7.87 (m, 2H), 7.61-7.54 (m, 2H), 3.91 (s, 3H).

¹³C NMR (101 MHZ, CDCl₃) δ 166.5, 131.9, 131.3, 129.2, 128.2, 52.4.

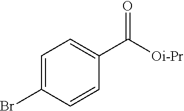

(1f) Isopropyl 4-bromobenzoate was obtained following the Starting Material General Procedure C. The spectroscopic data for this compound match those previously reported in the literature.

¹H NMR (400 MHZ, CDCl₃) δ 7.92-7.85 (m, 2H), 7.59-7.52 (m, 2H), 5.24 (hept, J=6.3 Hz, 1H), 1.36 (d, J=6.3 Hz, 6H).

¹³C NMR (101 MHZ, CDCl₃) δ 165.5, 131.7, 131.2, 129.9, 127.9, 68.9, 22.0.

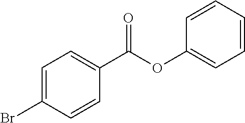 [SMILES: O=C(Oc1ccccc1)c1ccc(Br)cc1]

(1g) Phenyl 4-bromobenzoate was obtained following the Starting Material General Procedure C. The spectroscopic data for this compound match those previously reported in the literature.

¹H NMR (400 MHZ, CDCl₃) δ 8.07 (d, J=8.7 Hz, 2H), 7.70-7.63 (m, 2H), 7.48-7.39 (m, 2H), 7.32-7.26 (m, 1H), 7.24-7.17 (m, 2H).

¹³C NMR (101 MHZ, CDCl₃) δ 164.6, 150.9, 132.1, 131.8, 129.7, 129.0, 128.6, 126.2, 121.8.

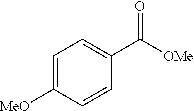 [SMILES: COC(=O)c1ccc(OC)cc1]

Methyl 4-methoxybenzoate was obtained following the Starting Material General Procedure C. The spectroscopic data for this compound match those previously reported in the literature.

¹H NMR (500 MHZ, CDCl₃) δ 8.01-7.97 (m, 2H), 6.94-6.90 (m, 2H), 3.89 (s, 3H), 3.86 (s, 3H).

¹³C NMR (126 MHZ, CDCl₃) δ 167.0, 163.5, 135.5, 131.7, 122.8, 113.8, 55.6, 52.0.

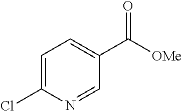 [SMILES: COC(=O)c1ccc(Cl)nc1]

Methyl 6-chloronicotinate was obtained following the Starting Material General Procedure B. The spectroscopic data for this compound match those previously reported in the literature.

¹H NMR (400 MHZ, CDCl₃) δ 9.00 (d, J=1.7 Hz, 1H), 8.24 (dd, J=8.3, 2.3 Hz, 1H), 7.42 (d, J=9.2 Hz, 1H), 3.96 (s, 3H).

¹³C NMR (101 MHZ, CDCl₃) δ 165.0, 155.8, 151.3, 139.7, 125.1, 124.3, 52.8.

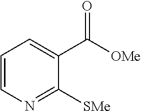 [SMILES: COC(=O)c1cccnc1SC]

Methyl 2-(methylthio) nicotinate was obtained following the Starting Material General Procedure B.

¹H NMR (400 MHZ, CDCl₃) δ 8.58 (dd, J=4.8, 1.9 Hz, 1H), 8.20 (dd, J=7.8, 1.9 Hz, 1H), 7.04 (dd, J=7.8, 4.8 Hz, 1H), 3.92 (s, 3H), 2.53 (s, 3H).

¹³C NMR (101 MHZ, CDCl₃) δ 165.0, 163.0, 152.1, 138.8, 123.0, 118.0, 52.4, 14.0.

IR (neat): 1711, 1553, 1277, 1233, 1129, 1057, 766 cm⁻¹.

HRMS: Calculated for C₈H₉NO₂S [M+H]⁺: 184.0427. Found: 184.0407.

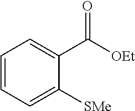 [SMILES: CCOC(=O)c1ccccc1SC]

Ethyl 2-thiomethylbenzoate was obtained following the Starting Material General Procedure C. The spectroscopic data for this compound match those previously reported in the literature.

¹H NMR (500 MHZ, CDCl₃) δ 7.99 (dd, J=7.8, 1.6 Hz, 1H), 7.45 (ddd, J=8.9, 7.3, 1.5 Hz, 1H), 7.25 (d, J=8.2 Hz, 1H), 7.16-7.11 (m, 1H), 4.37 (q, J=7.2 Hz, 2H), 2.44 (s, 3H), 1.38 (t, J=7.2 Hz, 3H).

¹³C NMR (126 MHZ, CDCl₃) δ 166.5, 143.3, 132.5, 131.4, 127.3, 124.5, 123.5, 61.1, 15.7, 14.4.

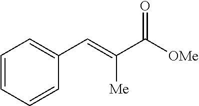 [SMILES: COC(=O)/C(C)=C/c1ccccc1]

Methyl 3-methyl-3-phenylacrylate was obtained following the Starting Material General Procedure C. The spectroscopic data for this compound match those previously reported in the literature.

¹H NMR (600 MHZ, CDCl₃) δ 7.70 (d, J=1.5 Hz, 1H), 7.40 (d, J=4.5 Hz, 4H), 7.34-7.31 (m, 1H), 3.82 (s, 3H), 2.13 (d, J=1.5 Hz, 3H).

¹³C NMR (126 MHz, CDCl₃) δ 139.1, 129.8, 128.5, 128.5, 52.2, 46.4, 14.2.

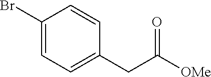 [SMILES: COC(=O)Cc1ccc(Br)cc1]

Methyl 2-(4-bromophenyl)acetate was obtained following the Starting Material General Procedure A. The spectroscopic data for this compound match those previously reported in the literature.

¹H NMR (600 MHZ, CDCl₃) δ 7.47-7.43 (m, 2H), 7.18-7.14 (m, 2H), 3.69 (s, 3H), 3.58 (s, 2H).

¹³C NMR (151 MHZ, CDCl₃) δ 171.6, 133.0, 131.8, 131.1, 121.3, 52.2, 40.6.

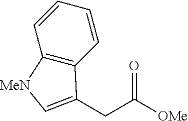 [SMILES: COC(=O)Cc1cn(C)c2ccccc12]

Methyl inodol-3-acetate was obtained following the Starting Material General Procedure A. Then, a 25 mL round-bottom flask was charged with NaH (2.0 equiv). Methyl inodol-3-acetate (1.0 equiv) was prepared as a solution in DMF (0.24 M). The solution was added dropwise to the round-bottom flask at 0° C. After approx. 15 min, Mel (1.4 equiv) was added dropwise to the reaction solution. The solution was warmed to room temperature and left to stir. Upon completion by TLC, the reaction solution was quenched with H₂O (approx. 10 mL) and diluted with CH₂Cl₂ (approx. 10 mL). The aqueous layer was washed with CH₂Cl₂, dried over Na₂SO₄, vacuum filtered, and concentrated in vacuo with the aid of a rotary evaporator. The crude residue was purified by automated column chromatography to afford methyl-indole-(N-methyl) 3-acetate. The spectroscopic data for this compound match those previously reported in the literature.

¹H NMR (400 MHZ, CDCl₃) δ 7.82 (d, J=7.9 Hz, 1H), 7.52 (d, J=8.2, Hz, 1H), 7.47 (s, 1H), 7.46-7.42 (m, 1H), 7.35 (t, J=7.7 Hz, 1H), 3.99 (s, 2H), 3.98 (s, 3H), 3.92 (s, 3H).

¹³C NMR (101 MHZ, CDCl₃) δ 172.7, 137.0, 127.9, 127.8, 121.9, 119.3, 119.1, 109.4, 106.9, 52.1, 32.8, 31.2.

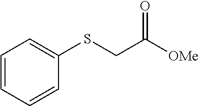 [SMILES: COC(=O)CSc1ccccc1]

Methyl 2-(phenylthiol)acetate was obtained following the Starting Material General Procedure A. The spectroscopic data for this compound match those previously reported in the literature.

¹H NMR (500 MHZ, CDCl₃) δ 7.43-7.39 (m, 2H), 7.34-7.28 (m, 2H), 7.26-7.21 (m, 1H), 3.72 (s, 3H), 3.66 (s, 2H).

¹³C NMR (151 MHZ, CDCl₃) δ 170.3, 135.0, 130.0, 129.2, 127.1, 52.7, 36.6.

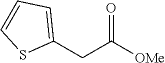

Methyl 2-thienylacetate was obtained following the Starting Material General Procedure A. The spectroscopic data for this compound match those previously reported in the literature.

¹H NMR (500 MHZ, CDCl₃) δ 7.22 (dd, J=5.1, 1.4 Hz, 1H), 6.97-6.93 (m, 2H), 3.85 (d, J=0.9 Hz, 2H), 3.73 (s, 3H).

¹³C NMR (151 MHZ, CDCl₃) δ 171.08, 135.12, 126.99, 126.96, 125.19, 52.41, 35.35.

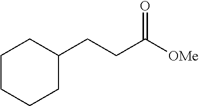 [SMILES: COC(=O)CCC1CCCCC1]

Methyl 3-cyclohexylpropionate was obtained following the Starting Material General Procedure C. The spectroscopic data for this compound match those previously reported in the literature.

¹H NMR (600 MHZ, CDCl₃) δ 3.66 (s, 3H), 2.34-2.27 (m, 2H), 1.69 (d, J=10.8 Hz, 4H), 1.64 (d, J=12.2 Hz, 1H), 1.52 (q, J=7.7 Hz, 2H), 1.26-1.18 (m, 3H), 1.18-1.09 (m, 1H), 0.93-0.83 (m, 2H).

¹³C NMR (151 MHz, CDCl₃) δ 174.8, 51.6, 37.4, 33.1, 32.5, 31.8, 26.7, 26.4.

What is claimed is:

1. A method for partial reduction of an ester to an aldehyde or nitrogen-containing compound, comprising:
preparing a reaction mixture comprising the ester, a Group IV metal catalyst, a silane, and an amine;
allowing the reaction mixture to undergo a partial reduction reaction; and
isolating an aldehyde or nitrogen-containing compound from the reaction mixture.

2. The method of claim 1, wherein the Group IV metal catalyst comprises zirconium, titanium, or hafnium.

3. The method of claim 1, wherein the Group IV metal catalyst is zirconocene dichloride or zirconocene hydrochloride.

4. The method of claim 1, wherein the silane is dimethoxy(methyl)silane (DMMS), diethoxy(methyl)silane (DEMS), or polymethylhydrosiloxane (PMHS).

5. The method of claim 1, wherein the amine is a primary or secondary amine.

6. The method of claim 1, wherein the amine is n-butylamine or piperidine.

7. The method of claim 1, wherein the ester is a polyester.

8. The method of claim 1, wherein the ester is an aryl ester, the Group IV metal catalyst is zirconocene dichloride or zirconocene hydrochloride, the silane is dimethoxy(methyl)silane (DMMS) or diethoxy(methyl)silane (DEMS), and the amine is n-butylamine.

9. The method of claim 1, wherein the ester is an aliphatic ester, the Group IV metal catalyst is zirconocene dichloride or zirconocene hydrochloride, the silane is diethoxy(methyl)silane (DEMS) or polymethylhydrosiloxane (PMHS), and the amine is piperidine.

10. The method of claim 1, wherein the reaction mixture further comprises anhydrous PhMe.

11. The method of claim 1, further comprising a step of placing the reaction mixture under a nitrogen environment prior to allowing the reaction mixture to undergo a partial reduction reaction.

12. The method of claim 1, further comprising a step of maintaining the reaction mixture at a temperature of 80° C. while the partial reduction reaction occurs.

13. The method of claim 1, wherein the nitrogen-containing compound is an imine, an enamine, a hydrazone, a N-heterocycle, or an amine.

14. A method for depolymerization of a polyester to an aldehyde or nitrogen-containing compound, comprising:
preparing a reaction mixture comprising the polyester, a Group IV metal catalyst, a silane, and an amine;
allowing the reaction mixture to undergo a partial reduction reaction; and
isolating an aldehyde or nitrogen-containing compound from the reaction mixture.

15. The method of claim 14, wherein the Group IV metal catalyst is zirconocene dichloride or zirconocene hydrochloride.

16. The method of claim 14, wherein the silane is dimethoxy(methyl)silane (DMMS), diethoxy(methyl)silane (DEMS), or polymethylhydrosiloxane (PMHS).

17. The method of claim 14, wherein the amine is a primary or secondary amine.

18. The method of claim 14, wherein the amine is n-butylamine or piperidine.

19. The method of claim 14, wherein the nitrogen-containing compound is an imine, an enamine, a hydrazone, a N-heterocycle, or an amine.
